(19) United States
(12) Patent Application Publication
(10) Pub. No.: US 2022/0267380 A1
(43) Pub. Date: Aug. 25, 2022

(54) Glycopeptides Increasing Lipid Synthesis

(71) Applicant: TFCHEM, Val De Reuil (FR)

(72) Inventors: Géraldine Deliencourt-Godefroy, Sainte Adresse (FR); Jocelyne Legoedec, Sotteville-Les-Rouen (FR)

(73) Assignee: TFCHEM, Val De Reuil (FR)

(21) Appl. No.: 17/627,347

(22) PCT Filed: Jul. 17, 2020
(86) PCT No.: PCT/EP2020/070339

§ 371 Date: Jan. 14, 2022

(30) Foreign Application Priority Data
Jul. 17, 2019 (EP) 19305945.8

(51) Int. Cl. C07K 9/00; A61Q 19/08; A61K 8/64; A61Q 7/00

(52) U.S. Cl. CPC C07K 9/001; A61Q 19/08; A61K 8/64; A61Q 7/00

(57) ABSTRACT

The present invention relates to a glycopeptide of the following formula I: or a tautomer, a stereoisomer or a mixture of stereoisomers thereof in any proportions, in particular a mixture of enantiomers, and particularly a racemate mixture, and/or a physiologically acceptable salt and/or solvate thereof, in which:—n represents an integer from 1 to 6,—m represents 0 or 1,10—p represents 0 or 1—R represents H, F, CH₃, CH₂F, or CH₂OH,—R₁, R₂ and R₃ represent, independently from one another, H, F, or OH,—R₄ represents a hydrogen, a halogen, or OH,—R₆ and R₇ represent, independently from each other, a hydrogen, a (C₁-C₆)alkyl, an aryl, or an aryl-(C₁-C₆)alkyl, and—R₈ represents H or a CO-(C₁-C₂₀)alkyl, useful for skin plumping and/or skin volumizing and/or skin densifying and/or wrinkle filling and/or skin or hair moisturizing and/or skin or hair relipiding and/or stimulation of hair growth and/or for the treatment of dry skin and/or atopic dermatitis and/or atopic eczema and/or psoriasis, due to its ability to increase lipid synthesis.

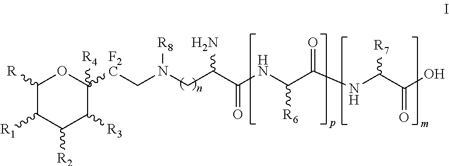

### TECHNICAL FIELD

The present invention relates to the use of glycopeptide derivatives in cosmetic or dermatological applications, in particular for skin plumping and/or skin volumizing and/or skin densifiying, and/or wrinkle filling and/or skin or hair moisturising and/or skin or hair relipiding and/or stimulation of hair growth and/or for the treatment of dry skin and/or atopic dermatitis and/or eczema and/or psoriasis.

### BACKGROUND

With age, the loss of skin elasticity and the degradation of adipose tissue lead to undesirable apparent effect on the body (hands, feet, buttocks, breasts, face) and notably on the face: appearance of lines and wrinkles, decrease of skin volume around the eyes and hollow cheeks. To reshape body, to fill expression lines and wrinkles, and to plump the skin, chirurgical fat injection (fat grafting or lipofilling) has been developed, and consist in restoring the volume of the skin, particularly the face, by the reinjection of fat removed from a rich fat site of the body. However, this technique currently used is expensive, can cause inflammatory reactions and needs to be redone several times for a satisfactory result.

In order to find new lipofilling method, scientists were interested in skin physiology and more particularly in adipose tissue and its components. Adipose tissue is predominantly composed of adipocytes and of others cells such as preadipocytes, fibroblasts or endothelial cells. Adipocytes are the site of lipid synthesis and storage, they are provided from the process of adipogenesis also called adipocyte differentiation in which preadipocytes developed into mature adipocytes (Eur. J. Cell Biol. 2013, 92, 229-236).

It has also been shown that fibroblasts and adipocytes are provided from common mesenchymal multipotent precursors (Exp. Dermatol. 2014, 23(9), 629-631). Thus, adipocyte cells could be generated by the differentiation of fibroblasts.

The stimulation of the adipogenesis and synthesis of lipid create an increase of adipocyte volume and therefore restore volume to the skin. That is why, compounds with an efficacy to increase adipocytes number and volume have been described for their ability to act as skin plumping and/or volumizing and/or densifying agents and/or wrinkle filling agents.

In addition, a decrease of lipid synthesis can create skin barrier abnormalities observed in dry skin (W098/10739), in atopic dermatitis, in eczema or in psoriasis (J. Invest. Dermatol. 1991, 96, 523-526, Skin Pharmacol. Physiol. 2015, 28, 42-55).

Moreover, it has also been proved that lipids and more particularly the cholesterol synthesis play a major role in hair biology. Thus, a decrease in lipid synthesis and particularly in cholesterol disturb hair cycle (J. Invest. Dermatol. 2010, 130(5), 1205-1207, J. Invest. Dermatol. 2010,130, 1237-48)

In the framework of the present invention, the inventors have surprisingly demonstrated an inducing activation of the signaling pathway of lipid synthesis and cholesterol synthesis at a transcriptional level in fibroblasts in the presence of glycopeptide derivatives.

Preparation of glycopeptide derivatives according to the present invention and their preservative/protective effect on human skin fibroblasts and human nasal epithelial cells in vitro under different stresses, such as starvation conditions, UV stress, oxidative stress or bacterial stress, were described in international application WO2015/140178. None of the results presented in this PCT application could have enable the one skilled in the art to anticipate the fact that the glycopeptide derivates according to the invention could stimulate the synthesis of lipids and thus could be used as skin plumping and/or volumizing and/or densifying agent and/or wrinkle filling agent and/or skin or hair moisturizing agent and/or skin or hair relipiding and/or hair growth stimulating agent, and/or in the treatment of dry skin, psoriasis, dermatitis atopic or eczema.

### SUMMARY OF THE INVENTION

According to a first aspect, the present invention relates to the use of a glycopeptide of the following formula I or I′:

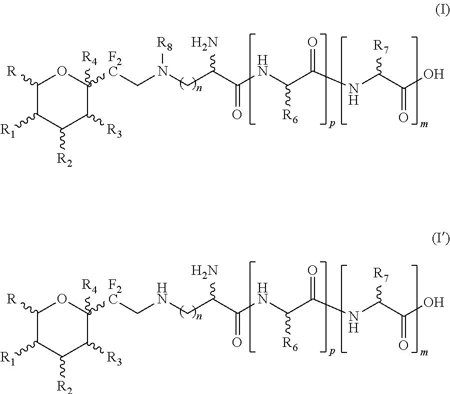

or a tautomer, a stereoisomer or a mixture of stereoisomers thereof in any proportions, in particular a mixture of enantiomers, and particularly a racemate mixture, and/or a physiologically acceptable salt and/or solvate thereof, in which:
n represents an integer from 1 to 6,
m represents 0 or 1,
p represents 0 or 1
R represents H, F, CH₃, CH₂F, or CH₂OH,
R₁, R₂ and R₃ represent, independently from one another, H, F, or OH,
R₄ represents a hydrogen, a halogen, or OH,
R₆ and R₇ represent, independently from each other, a hydrogen, a (C₁-C₆)alkyl, an aryl, or an aryl-(C₁-C₆)alkyl,
R₈ represents H or R₉, notably H, and
R₉ represents a CO-(C₁-C₂₀)alkyl (e.g. CO-(C₁-C₁₅)alkyl), for skin plumping and/or skin volumizing and/or skin densifying and/or wrinkle filling and/or skin or hair moisturizing and/or skin or hair relipiding and/or stimulation of hair growth. The invention relates also to a method for skin plumping and/or skin volumizing and/or skin densifying and/or wrinkle filling and/or skin or hair moisturizing and/or skin or hair relipiding and/or stimulation of hair growth comprising the administration, notably topically onto the skin (including the scalp skin for the stimulation of hair growth) or subcutaneously, of a glycopeptide of formula I or I′ as mentioned above, or a tautomer, a stereoisomer or a mixture of stereoisomers thereof in any proportions, in particular a mixture of enantiomers, and particularly a racemate mixture, and/or a physiologically acceptable salt and/or solvate thereof. The invention relates also to a glycopeptide of formula I or I′ as mentioned above, or a tautomer, a stereoisomer or a mixture of stereoisomers thereof in any proportions, in particular a mixture of enantiomers, and particularly a racemate mixture, and/or a physiologically acceptable salt and/or solvate thereof, for use for skin plumping and/or skin volumizing and/or skin densifying and/or wrinkle filling and/or skin or hair moisturizing and/or skin or hair relipiding and/or stimulation of hair growth.

The invention relates also to the use of a glycopeptide of formula I or I′ as mentioned above, or a tautomer, a stereoisomer or a mixture of stereoisomers thereof in any proportions, in particular a mixture of enantiomers, and particularly a racemate mixture, and/or a physiologically acceptable salt and/or solvate thereof, for the manufacture of a cosmetic or pharmaceutical (e.g. dermatological) composition intended for skin plumping and/or skin volumizing and/or skin densifying and/or wrinkle filling and/or skin or hair moisturizing and/or skin or hair relipiding and/or stimulation of hair growth.

The invention relates also to a method for skin plumping and/or skin volumizing and/or skin densifying and/or wrinkle filling and/or skin or hair moisturizing and/or skin or hair relipiding and/or stimulation of hair growth comprising the administration to a person in need thereof of an effective amount of a glycopeptide of formula I or I′ as mentioned above, or a tautomer, a stereoisomer or a mixture of stereoisomers thereof in any proportions, in particular a mixture of enantiomers, and particularly a racemate mixture, and/or a physiologically acceptable salt and/or solvate thereof.

The glycopeptide of formula I or I′ as mentioned above, or a tautomer, a stereoisomer or a mixture of stereoisomers thereof in any proportions, in particular a mixture of enantiomers, and particularly a racemate mixture, and/or a physiologically acceptable salt and/or solvate thereof, can be used or administered by means of a composition, in particular a cosmetic or dermatological composition, comprising said glycopeptide of formula I or I′ as mentioned above, or a tautomer, a stereoisomer or a mixture of stereoisomers thereof in any proportions, in particular a mixture of enantiomers, and particularly a racemate mixture, and/or a physiologically acceptable salt and/or solvate thereof, and at least one physiologically acceptable excipient.

According to a second aspect, the present invention relates to a glycopeptide of the following formula I or I′:

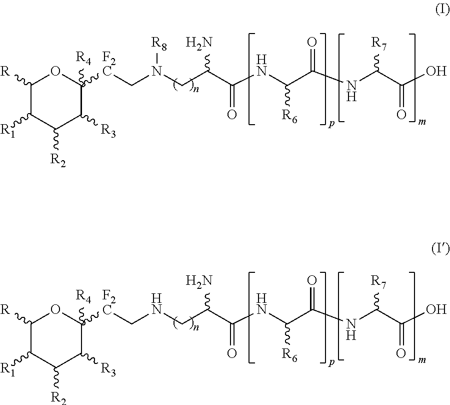

or a tautomer, a stereoisomer or a mixture of stereoisomers thereof in any proportions, in particular a mixture of enantiomers, and particularly a racemate mixture, and/or a physiologically acceptable salt and/or solvate thereof, in which:
n represents an integer from 1 to 6,
m represents 0 or 1,
p represents 0 or 1
R represents H, F, CH₃, CH₂F, or CH₂OH,
R₁, R₂ and R₃ represent, independently from one another, H, F, or OH,
R₄ represents a hydrogen, a halogen, or OH,
R₆ and R₇ represent, independently from each other, a hydrogen, a (C₁-C₆)alkyl, an aryl, or an aryl-(C₁-C₆)alkyl,
R₈ represents H or R₉, notably H, and
R₉ represents a CO-(C₁-C₂₀)alkyl (e.g. CO-(C₁-C₁₅)alkyl), for use in the treatment of dry skin and/or atopic dermatitis and/or atopic eczema and/or psoriasis.

The invention relates also to the use of a glycopeptide of formula I or I′ as mentioned above, or a tautomer, a stereoisomer or a mixture of stereoisomers thereof in any proportions, in particular a mixture of enantiomers, and particularly a racemate mixture, and/or a physiologically acceptable salt and/or solvate thereof, for the treatment of dry skin and/or atopic dermatitis and/or atopic eczema and/or psoriasis.

The invention relates also to the use of a glycopeptide of formula I or I′ as mentioned above, or a tautomer, a stereoisomer or a mixture of stereoisomers thereof in any proportions, in particular a mixture of enantiomers, and particularly a racemate mixture, and/or a physiologically acceptable salt and/or solvate thereof, for the manufacture of a cosmetic or pharmaceutical (e.g. dermatological) composition intended for the treatment of dry skin and/or atopic dermatitis and/or atopic eczema and/or psoriasis.

The invention relates also to a method for the treatment of dry skin and/or atopic dermatitis and/or atopic eczema and/or psoriasis comprising the administration to a person in need thereof of an effective amount of a glycopeptide of formula I or I′ as mentioned above, or a tautomer, a stereoisomer or a mixture of stereoisomers thereof in any proportions, in particular a mixture of enantiomers, and particularly a racemate mixture, and/or a physiologically acceptable salt and/or solvate thereof.

The glycopeptide of formula I or I′ as mentioned above, or a tautomer, a stereoisomer or a mixture of stereoisomers thereof in any proportions, in particular a mixture of enantiomers, and particularly a racemate mixture, and/or a physiologically acceptable salt and/or solvate thereof, can be used or administered by means of a composition, in particular a dermatological composition, comprising said glycopeptide of formula I or I′ as mentioned above, or a tautomer, a stereoisomer or a mixture of stereoisomers thereof in any proportions, in particular a mixture of enantiomers, and particularly a racemate mixture, and/or a physiologically acceptable salt and/or solvate thereof, and at least one physiologically acceptable excipient.

According to a third aspect, the present invention relates to a glycopeptide of the following formula I″:

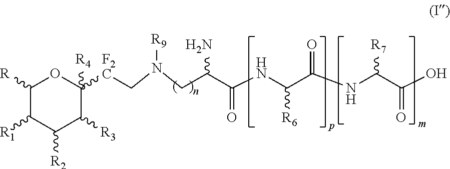

or a tautomer, a stereoisomer or a mixture of stereoisomers thereof in any proportions, in particular a mixture of enantiomers, and particularly a racemate mixture, and/or a physiologically acceptable salt and/or solvate thereof, in which:
n represents an integer from 1 to 6,
m represents 0 or 1,
p represents 0 or 1
R represents H, F, CH₃, CH₂F, or CH₂OH,
R₁, R₂ and R₃ represent, independently from one another, H, F, or OH,
R₄ represents a hydrogen, a halogen, or OH,
R₆ and R₇ represent, independently from each other, a hydrogen, a (C₁-C₆)alkyl, an aryl, or an aryl-(C₁-C₆)alkyl, and
R₉ represents a CO-(C₁-C₂₀)alkyl (e.g. CO-(C₁-C₅)alkyl).

According to a fourth aspect, the present invention relates to a cosmetic or dermatological composition comprising a glycopeptide of formula I″ as defined above and at least one physiologically acceptable excipient.

### DEFINITION

For the purpose of the invention, the term “physiologically acceptable” is intended to mean what is useful to the preparation of a cosmetic or pharmaceutical (e.g. dermatological) composition, and what is generally safe and non toxic, for a cosmetic or pharmaceutical (e.g. dermatological) use, notably in a mammal such as a human. The term “physiologically acceptable salt and/or solvate” is intended to mean, in the framework of the present invention, a salt and/or solvate of a compound which is physiologically acceptable, as defined above, and which possesses the cosmetic or pharmacological activity of the corresponding compound.

In the context of the present invention, the “physiologically acceptable salt” can be:

(1) an acid addition salt formed with an inorganic acid such as hydrochloric, hydrobromic, sulfuric, nitric and phosphoric acid and the like; or formed with an organic acid such as acetic, benzenesulfonic, fumaric, glucoheptonic, gluconic, glutamic, glycolic, hydroxynaphtoic, 2-hydroxyethanesulfonic, lactic, maleic, malic, mandelic, methanesulfonic, muconic, 2-naphtalenesulfonic, propionic, succinic, dibenzoyl-L-tartaric, tartaric, p-toluenesulfonic, trimethylacetic and trifluoroacetic acid and the like, or
(2) a salt formed when an acid proton present in the compound is either replaced by a metal ion, such as an alkali metal ion, an alkaline-earth metal ion, or an aluminium ion; or coordinated with an organic or inorganic base. Acceptable organic bases comprise diethanolamine, ethanolamine, N-methylglucamine, triethanolamine, tromethamine and the like. Acceptable inorganic bases comprise aluminium hydroxide, calcium hydroxide, potassium hydroxide, sodium carbonate, sodium hydroxide and the like. The salt can be more particularly a salt formed with an acid, such as hydrochloric acid or acetic acid.

In the context of the present invention, a “physiologically acceptable solvate” of a glycopeptide derivative of the present invention include conventional solvates such as those formed during the last step of the preparation of the compounds of the invention due to the presence of solvents. As an example, mention may be made of solvates due to the presence of water (these solvates are also called hydrates) or ethanol.

For the purpose of this invention, “tautomer” is intended to designate the various tautomer forms that the sugar of the glycopeptide according to the invention may assume, namely a pyranose (6-membered ring), furanose (5-membered ring) or linear (open form) form. However, for practical reasons, the sugar of the glycopeptide according to the invention is represented in the present description by its pyranose form. However, the compounds of the invention can assume various tautomer forms only when the radical R₄ represents an OH group, R₁ having also to represent an OH group in order that the glycopeptides of the invention can be in the furanose form.

Thus, for example, in the galactose series, the glycopeptides of the invention might appear under the following various forms (X═F):

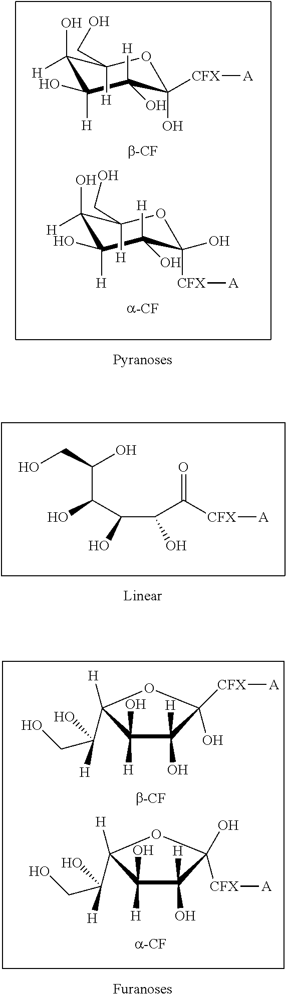

The group

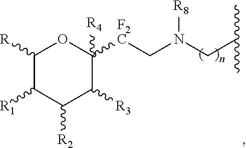

such as

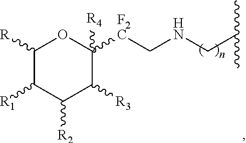

when R₄═R₁═OH can thus assume the following tautomer forms:
pyranose form

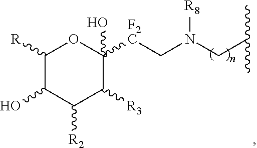

such as

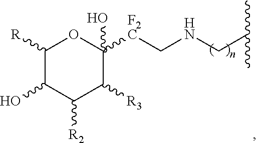

furanose form

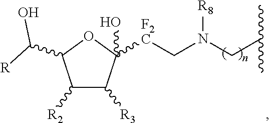

such as

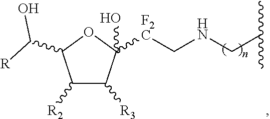

and
linear form:

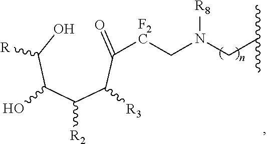

such as

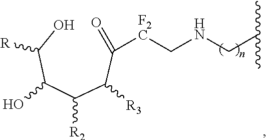

In the same way, the group

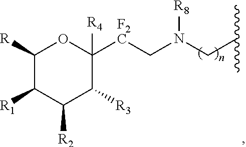

such as

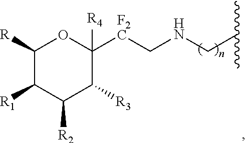

when R₄═R₁═OH can thus assume the following tautomer forms:
pyranose form:

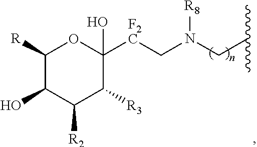

such as

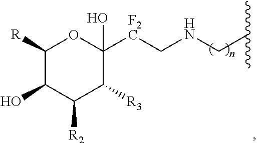

furanose form:

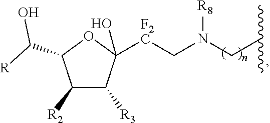

such as

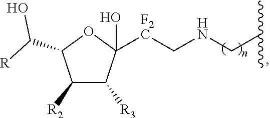

and
linear form

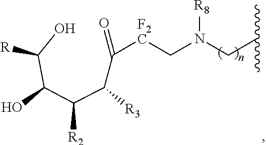

such as

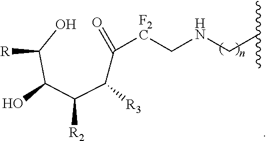

The anomeric carbon can appear in two different configurations in the closed pyranose and furanose forms.
The compounds of the invention can assume different tautomer forms which can be present in solution in equilibrium, with optionally a major tautomer form relatively to the other(s) tautomer form(s), or the compounds of the invention can assume only one tautomer form, such as only a pyranose form. This will depend notably on the nature of the medium, the temperature, the concentration of the compound, etc.

Within the meaning of this invention, “stereoisomers” is intended to designate diastereoisomers or enantiomers. These are therefore optical isomers. Stereoisomers which are not mirror images of one another are thus designated as “diastereoisomers,” and stereoisomers which are non-superimposable mirror images are designated as “enantiomers”.

Notably, the sugar moiety and the amino acid moieties of the compounds of the invention can belong to the D or L series.

A carbon atom bond to four non-identical sub stituents is called a “chiral centre”.

An equimolar mixture of two enantiomers is called a “racemate mixture”.

For the purpose of this invention, “rotamer”, also called “rotational isomer” is intended to designate conformational isomers that the glycopeptide according to the invention may assume, said conformational isomers being obtained by rotations about single bonds present in the molecule of glycopeptide. Contrary to stereoisomers, rotamers cannot be isolated since they are interconvertible by free rotation about single bonds.

The term “halogen” as used in the present invention refers to an atom of fluorine, bromine, chlorine or iodine. Advantageously, this is an atom of fluorine.

The term “(Cₓ-C_{y})alkyl” as used in the present invention refers to a saturated, linear or branched hydrocarbon chain comprising from x to y carbon atoms. Thus, for example, the term “(C₁-C₆)alkyl” as used in the present invention refers to a saturated, linear or branched hydrocarbon chain comprising from 1 to 6 carbon atoms, in particular the methyl, ethyl, n-propyl, isopropyl, n-butyl, iso-butyl, sec-butyl, tert-butyl, n-pentyl, n-hexyl groups. It can be in particular a methyl group.

The term “CO-(Cₓ-C_{y})alkyl” as used in the present invention refers to a (Cₓ-C_{y})alkyl group as defined above bound to the rest of the molecule by means of a carbonyl (CO) group. Thus, for example, the term “CO-(C₁-C₂₀)alkyl” as used in the present invention refers to a (C₁-C₂₀)alkyl group bound to the rest of the molecule by means of a carbonyl (CO) group, such as an acetyl or palmitoyl group.

The term “aryl”, as used in the present invention, refers to an aromatic hydrocarbon group comprising preferably 6 to 10 carbon atoms and comprising one or more fused rings, such as, for example, a phenyl or naphthyl group. Advantageously, it will be a phenyl group.

The term “aryl-(C₁-C₆)-alkyl” as used in the present invention refers to any aryl group as defined above, which is bound to the molecule by means of a (C₁-C₆)-alkyl group as defined above. In particular, it can be a benzyl group.

The terms “skin plumping”, “skin volumizing” and “skin densifying”, as used in the present invention, refers to the fact to reshape the skin and to increase volume of the skin, notably by increasing the adipose volume.

The term “wrinkle filling”, as used in the present invention, refers to the fact to restore the volume, fullness and smoothness of the skin in order to reduce or eliminate wrinkles, including expression lines, notably by increasing the adipose volume.

The term “skin or hair moisturising”, as used in the present invention, refers to the fact to increase the moisture content of the skin or the hair and to keep the skin soft, supple and smooth and to keep the hair soft, supple and shine, notably by increasing lipid (e.g. cholesterol) synthesis.

The term “skin or hair relipiding”, as used in the present invention, refers to the fact to increase the lipid content of the skin or the hair in order to restore the hydrolipidic film of the skin or the hair so as to keep the skin soft, supple and smooth and to keep the hair soft, supple and shine.

### DETAILED DESCRIPTION

### 1. Glycopeptide Derivatives

The glycopeptide according to the present invention has: the following formula I″:

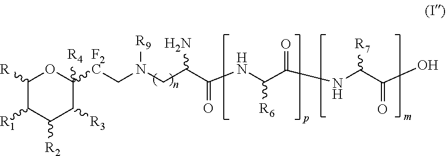

or the following formula I or I′:

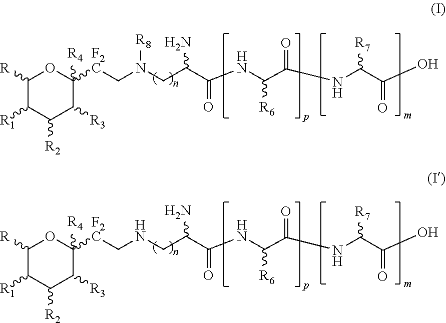

when used for skin plumping and/or skin volumizing and/or skin densifying and/or wrinkle filling and/or skin or hair moisturizing and/or skin or hair relipiding and/or stimulation of hair growth and/or for treating dry skin and/or atopic dermatitis and/or atopic eczema and/or psoriasis,
or a tautomer, a stereoisomer or a mixture of stereoisomers thereof in any proportions, in particular a mixture of enantiomers, and particularly a racemate mixture, and/or a physiologically acceptable salt and/or solvate thereof, in which:
n represents an integer from 1 to 6,
m represents 0 or 1,
p represents 0 or 1
R represents H, F, CH₃, CH₂F, or CH₂OH,
R₁, R₂ and R₃ represent, independently from one another, H, F, or OH,
R₄ represents a hydrogen, a halogen, or OH,
R₆ and R₇ represent, independently from each other, a hydrogen, a (C₁-C₆)alkyl, an aryl, or an aryl-(C₁-C₆)alkyl,
R₈ represents H or R₉, notably H, and
R₉ represents a CO-(C₁-C₂₀)alkyl (e.g. CO-(C₁-C₁₅)alkyl).

This glycopeptide according to the invention can be obtained in the form of a mixture of rotamers.

According to a particular embodiment, the sugar moiety of the glycopeptide of formula I, I′ or I″ of the present invention is in the galactose series, so that the glycopeptide according to the invention is advantageously a glycopeptide of the following formula (Ia), (Ib), (Ic), (Ia'), (Ib'), (Ic'), (Ia″), (Ib″) or (Ic″):

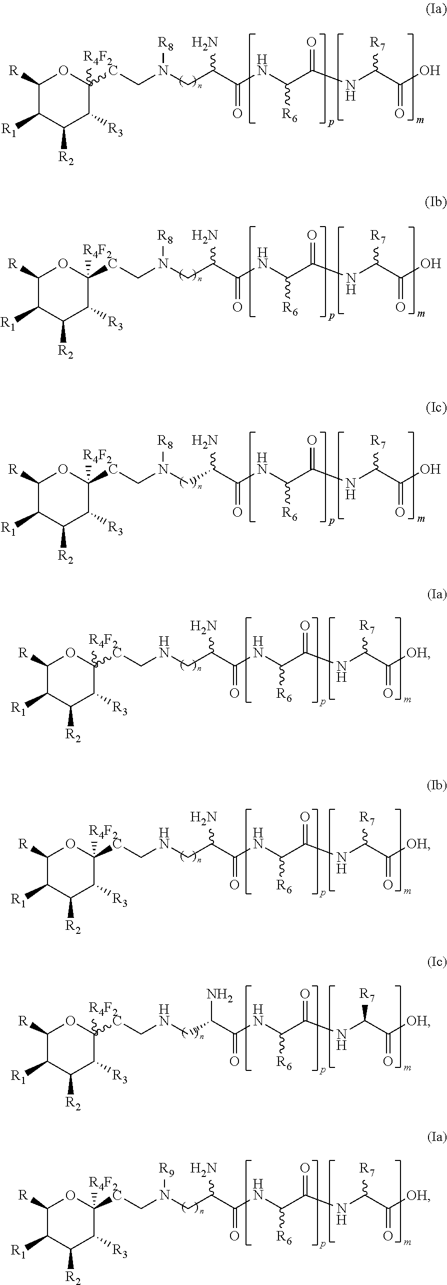

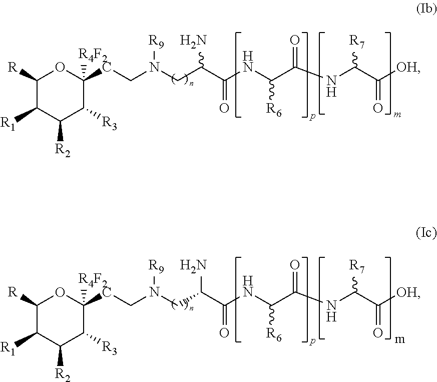

or a tautomer, and/or a physiologically acceptable salt and/or solvate thereof.

n represents 1, 2, 3, 4, 5 or 6. Advantageously, n represents an integer from 2 to 6, notably from 3 to 5, such as 4.

m and p each represent l, or m and p each represent 0, or one of m and p is 0 and the other is 1. In particular, m and p each represent 1.

Advantageously, R represents CH₂OH and R₁, R₂ and R₃ each represent OH.

Advantageously, R₄ represents OH.

In particular, R₆ and R₇ represent, independently from each other, H, CH₃, CH(CH₃)₂, CH₂CH(CH₃)₂, CH(CH₃)CH₂CH₃, or CH₂Ph. Preferably, R₆ and R₇ represent, independently from each other, a (C₁-C₆)alkyl, such as a methyl.

R₈ represents H or R₉. In particular, R₈ represents H, an acetyl or a palmitoyl group, notably H.

R₉ represents a CO-(C₁-C₂₀)alkyl, such as a CO-(C₁-C₁₅)alkyl), for example a CO-(C₁-C₆)alkyl. R₉ can be an acetyl or a palmitoyl group.

According to a first particular embodiment, n represents an integer from 2 to 6, i.e. 2, 3, 4, 5 or 6, notably 4, R represents a CH₂OH group and R₁, R₂ and R₃ represents OH.

According to a second particular embodiment, n represents an integer from 2 to 6, i.e. 2, 3, 4, 5 or 6, notably 4, R represents a CH₂OH group, and R₁, R₂, R₃ and R₄ represent OH.

According to a third particular embodiment, n represents an integer from 2 to 6, i.e. 2, 3, 4, 5 or 6, notably 4, R represents a CH₂OH group, R₁, R₂, R₃ and R₄ represent OH, and m and p each represent 1.

According to a fourth particular embodiment, n represents an integer from 2 to 6, i.e. 2, 3, 4, 5 or 6, notably 4, R represents a CH₂OH group, R₁, R₂, R₃ and R₄ represent OH, and m and p each represent 0.

According to a fifth particular embodiment, n represents an integer from 2 to 6, i.e. 2, 3, 4, 5 or 6, notably 4, R represents a CH₂OH group, R₁, R₂, R₃ and R₄ represent OH, and one of m and p is 0 and the other is 1.

According to a sixth particular embodiment, n represents an integer from 2 to 6, i.e. 2, 3, 4, 5 or 6, notably 4, R represents a CH₂OH group, R₁, R₂, R₃ and R₄ represent a OH group, and R₆ and R₇ represent a (C₁-C₆) alkyl such as methyl.

According to a seventh particular embodiment, n represents an integer from 2 to 6, i.e. 2, 3, 4, 5 or 6, notably 4, R represents a CH₂OH group, R₁, R₂, R₃ and R₄ represent a OH group, R₆ and R₇ represent a (C₁-C₆) alkyl such as methyl, and m and p each represent 1.

According to an eighth particular embodiment, n represents an integer from 2 to 6, i.e. 2, 3, 4, 5 or 6, notably 4, R represents a CH₂OH group, R₁, R₂, R₃ and R₄ represent a OH group, R₆ represents a (C₁-C₆) alkyl such as methyl, m is 1 and p is 0.

According to a ninth particular embodiment, n represents 4, R represents a CH₂OH group, R₁, R₂, R₃ and R₄ represent a OH group, R₆ and R₇ represent a (C₁-C₆) alkyl such as methyl, and m and p represent 1.

The compound of the present invention can be advantageously one of the following compounds 1 to 4, and notably is compound 1:

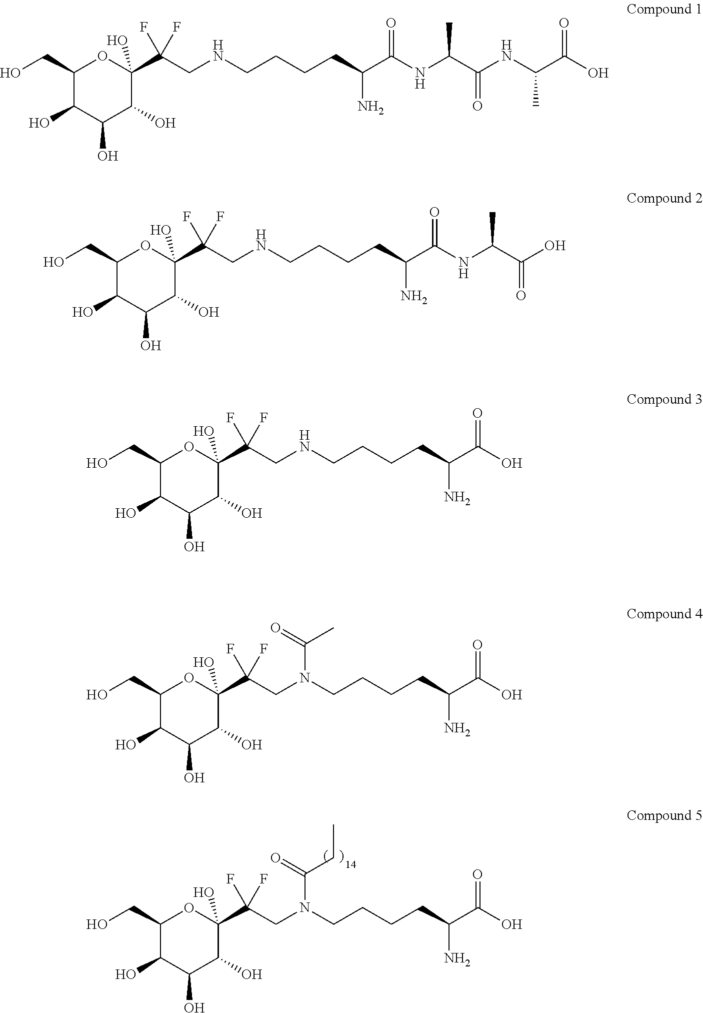

or a tautomer and/or a physiologically acceptable salt and/or solvate thereof.

Processes to prepare a glycopeptide according to formula I′ are disclosed in WO2015/140178. The same processes can be used to prepare the glycopeptides according to formula I″ with an additional step of substitution of the amino group (NH) in a of the CF₂ group with an R₉ group such a substitution step being well-known to the one skilled in the art.

### 2. Cosmetic and Dermatological Compositions

The glycopeptide derivative according to the present invention is used or administered by means of a cosmetic or dermatological composition comprising said glycopeptide and at least one physiologically acceptable excipient.

Such a composition is more particularly intended for a topical (e.g. transdermal) administration or a parenteral (e.g. subcutaneous) administration, preferably a topical administration, in particular on the skin, including the scalp skin, or an injection, in particular a subcutaneous injection.

Such a composition can thus be a solution, a dispersion, an emulsion, an oil, an ointment, a shampoo, a paste, a cream, a lotion, a milk, a foam, a gel, a suspension, a spray, a serum, a patch, a stick or a mask.

The composition of the invention may comprise one or several additive(s) as excipient(s), such as suspending agents, wetting agents, antioxidants, emollients, other moisturizing agents, thickening agents, chelating agents, buffering agents, fragrances, preservatives, pigments or colorants, opacifiers or mattifying agents. Such additives are conventional to those of skill in the art.

Examples of these additives are listed below as well as in the International Cosmetic Ingredient Dictionary and Handbook, eds. Wenninger and McEwen (The

Cosmetic, Toiletry, and Fragrance Assoc., Washington, D.C., 7@th Edition, 1997).

Suspending agents can be for example an alginate, sodium carboxymethyl cellulose, methyl cellulose, hydroxyl methyl cellulose, hydroxyl ethyl cellulose, hydroxylpropyl methyl cellulose, microcrystalline cellulose, a gum such as acacia, tragacanth or xanthan gum, gelatin, a carrageenan, polyvinyl pyrrolidone.

Wetting agents can be glycerin, propylene glycol or also nonionic surfactants such as a lecithin, a polysorbate or a poloxamer.

Antioxidants can be used to protect ingredients of the composition from oxidizing agents that are included within or come in contact with the composition. Examples of antioxidants include ascorbic acid, ascorbyl palmitate, citric acid, acetylcysteine, sulfurous acid salts (bisulfate, metabisulfite), sodium formaldehyde sulfoxylate, monothioglycerol, thiourea, butylated hydroxyanisole, butylated hydroxytoluene, potassium propyl gallate, octyl gallate, dodecyl gallate, phenyl-α-naphthyl-amine, and tocopherols such as α-tocopherol.

Emollients are agents that soften and smooth the skin. Examples of emollients include oils and waxes such as siloxanes such as dimethicone and derivatives thereof, microcrystalline wax, polyethylene, triglyceride esters such as those of castor oil, cocoa butter, safflower oil, corn oil, olive oil, cod liver oil, almond oil, palm oil, squalene, and soybean oil, acetylated monoglycerides, ethoxylated glycerides, fatty acids, alkyl esters of fatty acids, alkenyl esters of fatty acids, fatty alcohols, fatty alcohol ethers, ether-esters, lanolin and derivatives of lanolin, polyhydric alcohol esters, wax esters such as beeswax, vegetable waxes, phospholipids, sterols, isopropyl palmitate or glyceryl stearate.

A moisturising agent increases the moisture content of the skin and keeps it soft and smooth. It can be for example urea, an amino acid, lactic acid and its salts (such as sodium lactate), glycerol (also called glycerin), propylene glycol, butylene glycol, PEG (polyethylene glycol—such as PEG-4 to PEG-32), sorbitol, xylitol, maltitol, mannitol, polydextrose, collagen, elastin, hyaluronic acid and its salts (such as sodium or potassium salts), pectin, gelatin, chitosan, aloe vera, honey, etc.

Thickening agents are used to increase the viscosity and thickness of the composition. Examples of thickening agents include lipid thickening agents such as Cetyl Alcohol, Stearyl Alcohol, Myristyl Alcohol, Carnauba Wax, or Stearic acid; naturally derived thickening agents such as Cellulose derivatives like Hydroxyethylcellulose, Guar gum, Locust Bean Gum, Xanthan Gum, or Gelatin; mineral thickening agents such as Silica, Bentonite, or Magnesium Aluminum Silicate; synthetic thickening agents such as Carbomer; ionic thickening agents such as NaCl.

Chelating agents can be an ethylene diamine tetraacetic acid (EDTA) salt.

Buffering agents can be acetate, citrate, tartrate, phosphate, triethanolamine (TRIS).

Examples of fragrances or perfume include peppermint, rose oil, rose water, aloe vera, clove oil, menthol, camphor, eucalyptus oil, and other plant extracts. To eliminate certain odors from compositions, masking agents may be used.

Preservatives can be used to protect the composition from degradation. Examples of preservatives include phenol, cresol, chlorobutanol, phenoxyethanol, butylparaben, propylparaben, ethylparaben, methylparaben, propyl paraben, benzalkonium chloride, benzethonium chloride, benzoic acid, benzyl alcohol, and mixtures thereof such as liquipar oil. However, the composition of the present invention can be preservative free. Pigments or colorants are used to modify the color of the composition, such as to obtain a white composition.

Opacifiers, such as titanium oxide, are used in clear or transparent composition in order to render it opaque. The present invention can thus be clear or opaque according to the use or not of an opacifier.

Mattifying agents are ingredients that make the skin matt, which prevent it from shining. It can be for example talc, silica, rice powder, or a mixture thereof, notably in a micronized form.

The one skilled in the art will be able to adapt the amount of glycopeptide according to the invention in the cosmetic or dermatological composition in order to obtain the desired effect.

### 3. Cosmetic and Dermatological Applications

A glycopeptide derivative according to the present invention or a cosmetic or dermatological composition according to the present invention containing such a glycopeptide derivative is useful for skin plumping and/or skin volumizing and/or skin densifying and/or wrinkle filling and/or skin or hair moisturising and/or skin or hair relipiding and/or stimulation of hair growth.

Indeed, such effects can be obtained with the glycopeptide derivatives according to the invention due to their activity of increasing the volume of adipose tissue and the synthesis of lipids, such as cholesterol.

A glycopeptide derivative according to the present invention or a cosmetic or dermatological composition according to the present invention containing such a glycopeptide derivative is also useful for the treatment of dry skin and/or atopic dermatitis and/or eczema and/or psoriasis.

Indeed, as reported in the literature, such pathologies are associated with a decrease of lipid synthesis leading to a skin barrier impairment. It has been demonstrated that the glycopeptide derivatives according to the invention are useful in lipid synthesis so that such compounds can be used in the treatment of these pathologies.

The present invention is illustrated by the following non-limitative examples.

### FIGURES

FIGS. 1, 2, 3, 4 and 5 represent photographs of microscopic observations of normal human dermal fibroblasts (NHDF) for the control (FIG. 1 and FIG. 4) or after treatment with 10 mg/mL of compound 1 (FIG. 2) or with 20 mg/mL of compound 1 (FIG. 3) or with 13 mg/ml of compound 4 (FIG. 5).

FIG. 6 represents the synthesis scheme of compounds 4 and 5.

### EXAMPLES

The following abbreviations have been used in the examples:

DCE: Dichloroethane

DCM: Dichloromethane

DIEA: N,N-Diisopropylethylamine

NMR: Nuclear Magnetic Resonance

PTFE: Polytetrafluoroethylene

THF: Tetrahydrofuran

### 1. Synthesis of the Compounds According to the Invention

Compounds 1-3, in the form of their hydrochloride salts, have been prepared as reported in WO2015/140178. The base form has been obtained according to the following protocol:

Amberlite® IRA-67 (previously washed with water, 17.0 g) was added to a solution of the hydrochloride salt (12.3 mmol, 1 eq) in water (325 mL). The solution was stirred for 1 h 30 at room temperature. The pH of the solution was measured (pH=7.0) and the mixture was filtered (0.2 μm, HPTFE). The filtrate was then freeze-dried to afford the base form of compounds 1-3.

Compounds 4-5 have been prepared according to the synthesis scheme presented on FIG. 6. The synthesis protocols are detailed below:

### Synthesis of Compound C

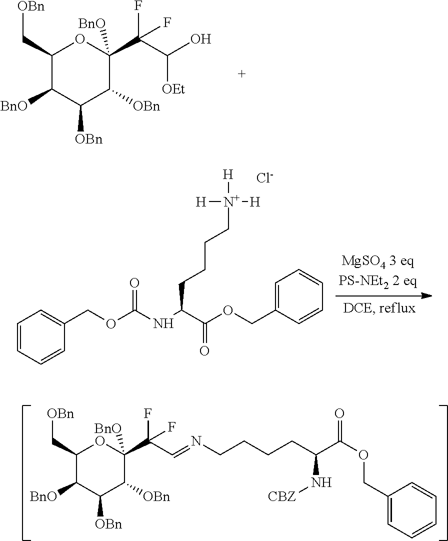

The synthesis of compounds A and B is described in WO2015/140178.

To a solution of compound B (89.5 g, 119 mmol, 1 eq) in anhydrous DCE (2.37 L) under inert atmosphere were sequentially added MgSO₄ (42.8 g, 356 mmol, 3 eq), compound A (48.23 g, 119 mmol, 1 eq) and diethylaminomethyl-polystyrene (˜3.2 mmol/g loading, 94.3 g, 237 mmol, 2 eq). The reaction was refluxed until ¹⁹F NMR showed complete conversion. The mixture was then cooled to room temperature and rapidly filtered over a pad of Celite® under a nitrogen stream. The resulting solution of the intermediate imine was transferred into a round-bottom flask and was used in the next step without purification.

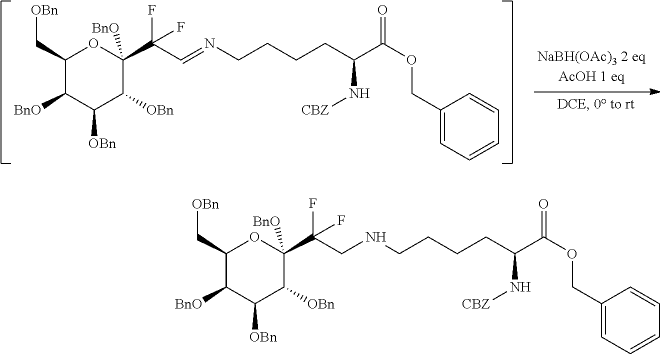

Sodium triacetoxyborohydride (51.5 g, 236 mmol, 2 eq) was added portion wise to a cold (0° C.) solution of the intermediate imine in DCE under inert atmosphere. Acetic acid (7.03 mL, 118 mmol, 1 eq) was then added dropwise to the mixture. The reaction mixture was stirred 30 min at 0° C. before being warmed to room temperature and stirred for 16 h. A solution of NaHCO₃ (saturated aqueous solution, 400 mL) was added and the mixture was vigorously stirred for 90 min. The mixture was then extracted with DCM (3×300 mL). The combined organic layer was then washed with brine, dried over sodium sulfate, filtered and concentrated. The residue was purified by flash chromatography (toluene/acetone 95:5 to 75:25) to afford compound C (65.6 g, 52% yield).

¹⁹Fdec NMR (CDCl₃, 282.5 MHz): −110.0 (d, 258 Hz, 1 F); −111.0 (d, 258 Hz, 1 F).

Mass (ESI⁺): 1063.4 [M+H]+.

### Synthesis of Compound D

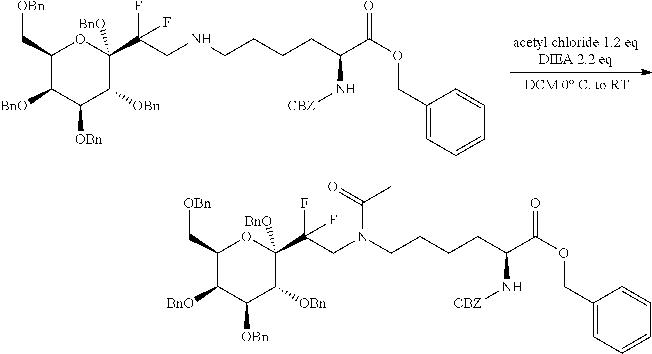

A solution of acetyl chloride (0.64 mL, 9.02 mmol, 1.2 eq) in DCM (50.0 mL) was added dropwise to a mixture of compound C (8.00 g, 7.52 mmol, 1 eq) and DIEA (2.74 mL, 16.6 mmol, 2.2 eq) in anhydrous DCM (50.0 mL) under inert atmosphere. The resulting solution was stirred at 25° C. for 24 h.

NH₄Cl (saturated aqueous solution) was added and the aqueous layer was extracted with DCM (3×150 mL). The combined organic layer was then washed with brine, dried over sodium sulfate, filtered and concentrated. The crude residue was purified by flash chromatography (AIT cartridge, 80 g, SiOH, cyclohexane/ethyl acetate 100:0 to 60:40) to afford compound D (6.58 g, 79% yield) as a white solid.

¹⁹F NMR (CDCl₃, 282.5 MHz): Compound D is present in the form of 2 rotamers. Form 1 (54%): −108.3 (brdd, 255 Hz, 30 Hz, 1 F); -112.3 (brdd, 256 Hz, 30 Hz, 1 F). Form 2 (46%): −110.2 (ddd, 259 Hz, 22 Hz, 10 Hz, 1 F); −111.3 (259 Hz, 22 Hz, 10 Hz, 1 F).

Mass (ESI+): 1105.5[M+H]⁺; 1127.5 [M+Na]⁺; 1143.5 [M+K]⁺

### Synthesis of compound E

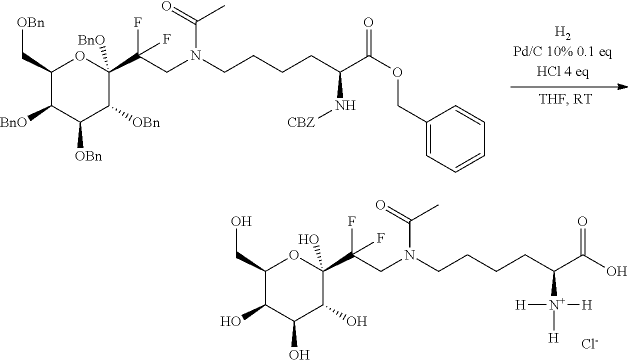

Palladium on carbon (loading 10 wt. %, support activated carbon, 0.63 g, 0.60 mmol, 0.1 eq) was added to a solution of compound D (6.58 g, 5.95 mmol, 1 eq) in THF (230 mL), previously degassed with nitrogen. A solution of HCl (2M in water, 11.9 mL, 23.81 mmol, 4 eq) was then added. The mixture was placed under hydrogen atmosphere and was stirred for 18 h. The reaction was degassed with nitrogen prior to be filtered (0.45 μm, polyamide) to remove the palladium residues. The filter was washed with a mixture of THF and water and the combined solution was concentrated to remove the THF. The residue was then diluted with water and the solution was freeze dried to afford compound E (2.78 g, 100%) as an amorphous off-white solid.

¹⁹Fdec NMR (MeOD, 282.5 MHz): Compound E is present under 4 major forms.

Form 1 (53%): −117.2 (d, 250 Hz, 1 F); −119.2 (d, 250 Hz, 1 F).

Form 2 (21%): −116.6 (d, 251Hz, 1F); −118.3 (d, 251 Hz, 1 F).

Form 3 (18%): −116.4 (d, 250Hz, 1F); −117.3 (d, 250 Hz, 1 F).

Form 4 (8%): −114.9 (d, 252Hz, 1F); −116.1 (d, 252 Hz, 1 F).

Mass (ESI+): 431.2[M+H]⁺ of NH₂ form

### Synthesis of compound 4

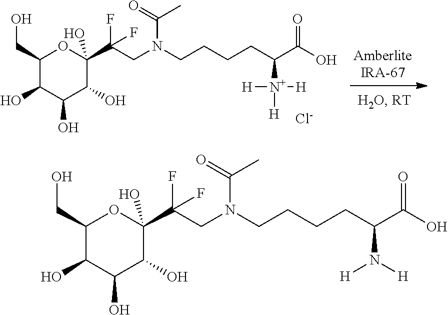 [SMILES: CC(=O)N(CCCC[C@H]([NH3+])C(=O)O)CC(F)(F)[C@]1(O)O[C@H](CO)[C@H](O)[C@H](O)[C@H]1O ; [Cl-] ; CC(=O)N(CCCC[C@H](N)C(=O)O)CC(F)(F)[C@]1(O)O[C@H](CO)[C@H](O)[C@H](O)[C@H]1O]

Amberlite® IRA-67 (previously washed with water, 13.0 g) was added to a solution of

compound E (2.78 g, 5.955 mmol, 1 eq) in water (156.6 mL). The solution was stirred for 1 h 30 at room temperature. The pH of the solution was measured (pH=7.0) and the mixture was filtered (0.2 μm, H-PTFE). The filtrate was then freeze-dried to afford compound 4 (2.39 g, 93% yield) as a white powder.

¹⁹F dec NMR (D₂O, 282.5 MHz): Compound 4 is present under 4 major forms.

Form 1 (32%): −116.0 (d, 250 Hz, 1 F); −118.1 (d, 250 Hz, 1 F).

Form 2 (29%): −115.4 (d, 250 Hz, 1 F); −117.2 (d, 250 Hz, 1 F).

Form 3 (22%): −115.4 (d, 252 Hz, 1 F); −116.3 (d, 252 Hz, 1 F).

Form 4 (17%): −114.4 (d, 252 Hz, 1 F); −115.5 (d, 252 Hz, 1 F).

Mass (ESI+): 431.2 [M+H]⁺

### Synthesis of Compound F

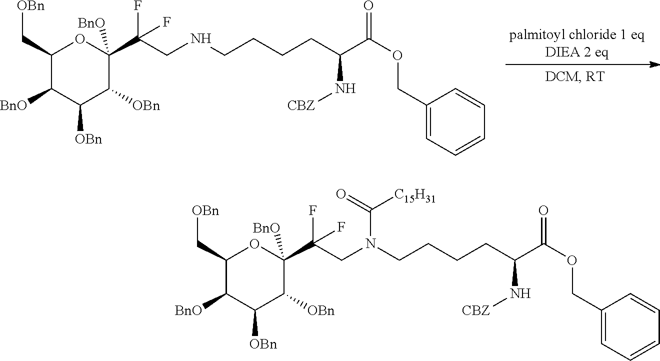 [C₁₅H₃₁═(CH₂)₁₄CH₃]

A solution of palmitoyl chloride (1.15 mL, 3.76 mmol, 1 eq) in anhydrous DCM (25 mL) was added dropwise to a mixture of compound C (4.0 g, 3.75 mmol, 1 eq) and DIEA (1.25 mL, 7.5 mmol, 2 eq) in anhydrous DCM (25 mL) under inert atmosphere. The reaction mixture was stirred at room temperature for 24 h. A solution of NH₄Cl (saturated aqueous solution) was then added. The aqueous layer was extracted with DCM (3×) and the combined organic layer was washed with brine, dried over sodium sulfate, filtered and concentrated. The crude residue was purified by flash chromatography (cyclohexane/ethyl acetate 100:0 to 55:45) to afford compound F (4.65 g, 95% yield) as a yellowish gel.

¹⁹F NMR (CDCl₃, 282.5 MHz): Compound F is present in the form of 2 rotamers.

Form 1 (56%): −108.6 (dd, 255 Hz, 32 Hz, 1 F); −112.6 (dd, 255 Hz, 27 Hz, 1 F). Form 2 (44%): −110.0 (ddd, 258 Hz, 27 Hz, 9 Hz, 1F); -112.6 (258 Hz, 27 Hz, 9 Hz, 1 F).

Mass (ESI⁺): 1301.7 [M+H]+; 1323.7 [M+Na]+; 1339.7 [M+K]+

### Synthesis of Compound G

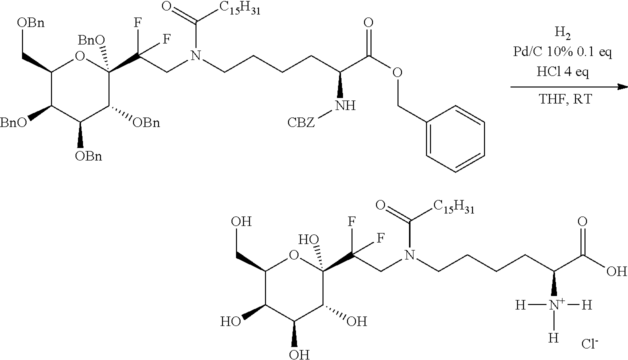

Palladium on carbon (loading 10 wt. %, support activated carbon, 0.38 g, 0.36 mmol, 0.1 eq) was added to a solution of compound F (4.65 g, 3.57 mmol, 1 eq) in THF (140 mL), previously degassed with nitrogen. A solution of HCl (2M in water, 7.15 mL, 14.3 mmol, 4 eq) was then added. The mixture was placed under hydrogen atmosphere and was stirred for 18h. The reaction was degassed with nitrogen prior to be filtered (0.45 μm, polyamide) to remove the palladium residues. The filter was washed with a mixture of THF and water and the combined solution was concentrated to remove the THF. The residue was then diluted with water and the solution was filtered (0.2 nm, H-PTFE) before being freeze dried to afford compound G (2.25 g, 95%) as a white powder.

¹⁹ F dec NMR (MeOD, 282.5 MHz): Compound G is present under 4 major forms.

Form 1 (53%): −119.5 (d, 249 Hz, 1 F); −117.6 (d, 249 Hz, 1 F).

Form 2 (23%): −117.7 (d, 250 Hz, 1 F); −116.8 (d, 250 Hz, 1 F).

Form 3 (18%): −118.3 (d, 250 Hz, 1 F); −116.4 (d, 250 Hz, 1 F).

Form 4 (6%): −116.4 (d, 252 Hz, 1 F); −114.9 (d, 252 Hz, 1 F).

Mass (ESI+): 627.4 [M+H]⁺of NH₂ form; 649.4 [M+Na]⁺NH₂ form

### Synthesis of compound 5

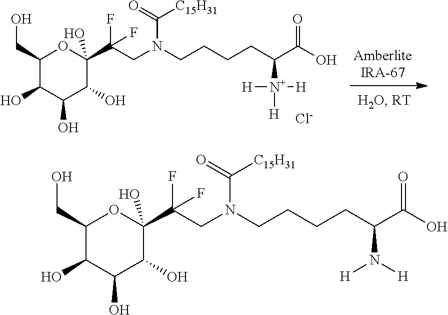

Amberlite® IRA-67 (previously washed with water, 7.3 g) was added to a solution of compound G (2.25 g, 3.39 mmol, 1 eq) in water (89 mL). The solution was stirred for 1 h 30 at room temperature. The pH of the solution was measured (pH=7.0) and the mixture was filtered (0.2 nm, H-PTFE). The filtrate was then freeze-dried to afford compound 5 (1.97 g, 93% yield) as a white powder.

¹⁹F dec NMR (MeOD, 282.5 MHz): Compound 5 is present under 4 major forms.

Form 1 (58%): −117.5 (d, 249 Hz, 1 F); −119.63 (d, 249 Hz, 1 F).

Form 2 (28%): −116.8(d, 250 Hz, 1 F); −118.6(d, 250 Hz, 1 F).

Form 3 (8%): −114.9 (d, 252 Hz, 1 F); −116.4 (d, 252 Hz, 1 F).

Form 4 (5%): −116.8 (d, 249 Hz, 1F); −117.7 (d, 249 Hz, 1 F).

Mass (ESI+): 627.4 [M+H]⁺; 649.4 [M+Na]⁺

### 2. Effects of Compound 1 and Compound 4 on Human Dermal Fibroblasts in Culture: Microscopic Observations

### Materials and Methods

Normal human dermal fibroblasts (NHDF) were grown with Dulbecco's Modified Eagle

Medium (DMEM) supplemented with Fetal Calf Serum (FCS) 10%, antibiotics (Penicillin 50 U/ml—Streptomycin 50 μg/ml) and L-Glutamine 2 mM final. Cells were grown in 37° C. and 5% CO₂ incubator.

Fibroblasts were seeded in 24-well plates and cultured in culture medium for 24 hours. The medium was then replaced by assay medium and cells were incubated for further 24 hours. For the assay, the medium was replaced by assay medium containing or not (control) the test compound at different concentrations. After 48 h the morphological observations were evaluated.

### Results

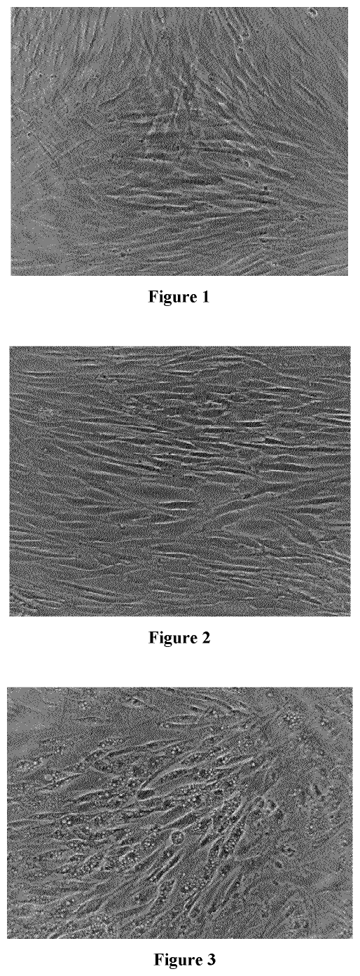

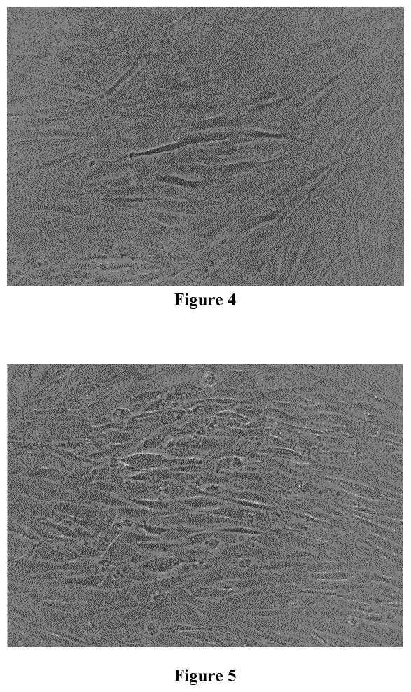

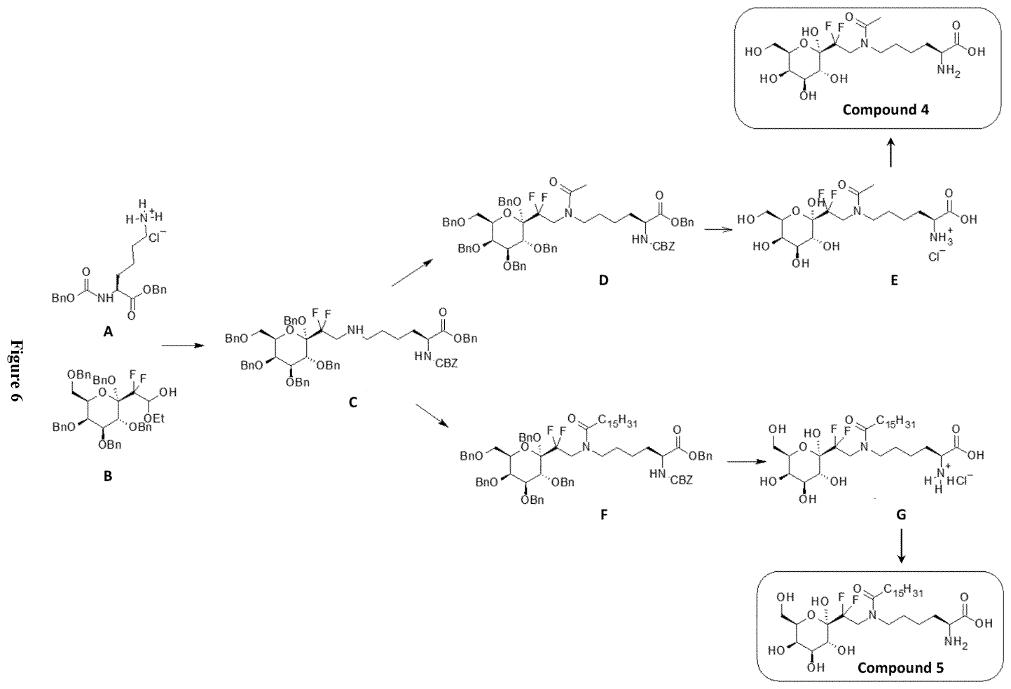

The effect of the compound 1 at 10 mg/ml and 20 mg/ml on fibroblasts culture was observed and compared to untreated cells. The representative images are reported in FIG. 1 (control), FIG. 2 (treatment at 10 mg/ml) and FIG. 3 (treatment at 20 mg/ml). Compared to untreated cells (control, FIG. 1), no effect has been shown with the tested compound used at 10 mg/ml (FIG. 2), whereas at 20 mg/ml (FIG. 3), compound 1 has induced the formation of lipid vesicles in fibroblasts. The effect of the compound 4 at 13 mg/ml on fibroblasts culture was observed and compared to untreated cells. The representative images are reported in FIG. 4 (control) and FIG. 5 (treatment at 13 mg/ml).

Compared to untreated cells (control, FIG. 4), the tested compound 4 used at 13 mg/ml (FIG. 5) induces the formation of lipid vesicles in fibroblasts and cell swelling.

### 3. Effects of Compound 1, 2, 3 and 4 on Gene Expression in Human Dermal Fibroblasts. Human«Full Transcriptome»Analysis Using Affymetrix Microarray

In the present study, the transcriptional effects (modulation of gene expression) of compounds 1, 2, 3 and 4 were evaluated on normal human dermal fibroblasts (NHDF) under basal conditions.

More specifically, the comparative analysis of the different transcriptomic profiles was performed using an Affymetrix GeneAtlas platform and the human “full transcriptome” U219 chip, which includes 36,000 transcripts and variants.

### Materials and Methods

### Gene Screening Assay

Fibroblasts were seeded in 24-well plates and cultured in culture medium for 24 hours. The medium was then replaced by assay medium and cells were incubated for further 24 hours. The medium was replaced by assay medium containing or not (control) the test compounds at different concentrations and the cells were preincubated for 24 hours. All experimental conditions were performed in triplicate. At the end of incubation, the culture supernatants were removed and the cells were washed in a phosphate buffered saline (PBS) solution and immediately frozen at −80° C.

### Differential Expression Analysis

Before RNA extraction, the replicates were pooled. Total RNA was extracted from each sample using TriPure Isolation Reagent® according to the supplier's instructions. The amount and quality of total RNA were evaluated for all samples using capillary electrophoresis (Bioanalyzer 2100, Agilent technologies). From each RNA, a labeled and amplified anti-sens RNA (aRNA) was obtained using GeneChip 3′IVT PLUS Kit (Affymetrix). For each labeled and amplified aRNA sample the profiles were evaluated before and after fragmentation using capillary electrophoresis (Bioanalyzer 2100, Agilent technologies). Hybridization of fragmented aRNA onto Affymetrix® U219 chip (36,000 transcripts and variants) was performed in the GeneAtlas™ fluidics Affymetrix® hybridization station for 20 hours at 45° C. U219 chip was analyzed using the GeneAtlas™ Imaging station (Affymetrix®—resolution 2 μm) to generate fluorescence intensity data.

### Data Management and Result Presentation

Expression Console and Quality controls: data were normalized with the Expression Console (Affymetrix®) software using RMA algorithm. Then a quality control of the labeling and the hybridization was performed. Hybridization and labeling steps successfully passed the quality controls for these experiments.

Data reduction, Excel file description: once normalized with Expression Console, data were transferred into a Microsoft Excel® file in order to go further into data reduction.

Calculation and tools were added in order to rank and sort data, and finally to support data interpretation. Detection thresholds in terms of fold change were defined and applied on normalized data.

| Fold Change | Arbitrary classification of observed effects |
|---|---|
| ≥2 | Upregulated probes (UP) |
| ≤0.5 | Downregulated probes (DR) |

Results are considered and presented or commented per gene (and not probe). A probe set is a collection of probes designed to interrogate a given sequence of a gene. For data interpretation, the most important relative expression value obtained with one probe is considered to be representative of the corresponding gene. The file contains the following data:
Relative expression (RE) for each sample,
Fold change calculation,
Gene information.

Identification of the biological processes involved: The list of significantly modulated genes was transferred in the online database DAVID (Database for Annotations, Visualization and Integrative Discovery: http://david.abcc.ncifcrf.gov/) for a functional analysis (Genome Biology 2007, 8:R183, Nucleic Acids Research, 2009, Vol. 37, No. 1 1-13). Gene Ontology database has been more specifically used for the data interpretation. DAVID functional annotation part was used to cluster modulated genes into significant biological processes. This analysis does not take into account the trend (UR or DR) or the signal intensity but only identifies the biological functions implicated in the comparison of interest. DAVID database uses the Gene Ontology consortium (http://www.geneontology.org) vocabularies (GO terms) to describe gene products in terms of their associated biological processes. Among them, only biological processes with p-value≤0.05 were taken into account.

Signal transduction pathway analysis: the results were then processed with IPA (Ingenuity Pathway Analysis, Qiagen®) software to identify signal transduction pathways modulated by each treatment. This software takes into account the Fold Change values of each gene and, when there is enough information, the direction of modulation of the signal transduction pathways can be identified. The relevance of the effect of each treatment on a given pathway was quantified by z-score. The z-score predicts the directional change on that effect.

| z-score | Predicted Activation State |
|---|---|
| >0 | Increased |
| <0 | Decreased |

### Results

### Identification of Biological Process Involved

The gene modulations of NHDF treated with compound 1 (10 mg/ml), compound 3 (10 mg/ml) and compound 4 (10 mg/ml) vs control were analysed to cluster modulated genes into significant biological processes (p-value≤0.05).

The table 1 below shows that the main biological processes involved with test compound 1, are the lipid metabolic process and the cholesterol biosynthetic process.

The table 2 below shows that the main biological processes involved with test compound 3, are the lipid metabolic process and the cholesterol biosynthetic process.

The table 3 below shows that the main biological processes involved with test compound 4, are the lipid metabolic process and the cholesterol biosynthetic process but also the ceramide metabolic process.

TABLE 1
Identification of the biological processes involved in NHDF
and stimulated by compound 1 (10 mg/ml)
Test compound 1 (10 mg/ml) versus Control

| Term | Count | % | p-value | Genes |
|---|---|---|---|---|
| Lipid | 13 | 0.01792189 | 5.96E−04 | SREBF1, CHKA, LDLR, |
| metabolic | | | | FADS1, ABHD5, |
| process | | | | PTGS1, ABHD4, |
| | | | | HMGCS1, FADS2, |
| | | | | ACAT2, APOD, |
| | | | | CH25H, LRP8 |
| Cholesterol | 14 | 0.01930049 | 3.83E−12 | EBP, MSMO1, MVD, |
| biosynthetic | | | | CYP51A1, HMGCR, |
| process | | | | DHCR7, INSIG1, |
| | | | | FDPS, HMGCS1, |
| | | | | LSS, MVK, IDI1, |
| | | | | HSD17B7, DHCR24 |

TABLE 2
Identification of the biological processes involved in NHDF
and stimulated by compound 3 (10 mg/ml)
Test compound 3 (10 mg/ml) versus Control

| Term | Count | % | p-value | Genes |
|---|---|---|---|---|
| Lipid | 14 | 0.029255653 | 2.24E−06 | SREBF1, CHKA, |
| metabolic | | | | PLA2G15, G6PD, |
| process | | | | APOD, PTGDS, |
| | | | | LDLR, TPP1, FADS1, |
| | | | | HMGCS1, ABHD4, |
| | | | | FADS2, LRP8, |
| | | | | ASAH1 |
| Cholesterol | 9 | 0.018807205 | 1.67E−07 | TM7SF2, EBP, G6PD, |
| biosynthetic | | | | MVD, DHCR7, |
| process | | | | INSIG1, HMGCS1, |
| | | | | LSS, DHCR24 |

TABLE 3
Identification of the biological processes involved in NHDF
and stimulated by compound 4 (10 mg/ml)
Test compound 4 (10 mg/ml) versus Control

| Term | Count | % | p-value | Genes |
|---|---|---|---|---|
| Lipid | 19 | 0.0134753 | 6.64E−04 | SREBF1, CHKA, SC5D, |
| metabolic | | | | PLA2G15, LDLR, FADS1, |
| process | | | | ABHD4, HMGCS1, |
| | | | | FADS2, ABHD3, |
| | | | | GPCPD1, ASAH1, |
| | | | | G6PD, PTGDS, APOD, |
| | | | | TPP1, LRP8, NR2F2, PC |
| Cholesterol | 11 | 0.0078015 | 9.91E−06 | TM7SF2, EBP, |
| biosynthetic | | | | G6PD, MVD, CYP51A1, |
| process | | | | DHCR7, INSIG1, |
| | | | | HMGCS1, LSS, |
| | | | | HSD17B7, |
| | | | | DHCR24 |
| Ceramide | 7 | 0.004964574 | 1.04E−04 | PPP2R1A, CLN3, |
| metabolic | | | | PLA2G15, |
| process | | | | HTRA2, COL4A3BP, |
| | | | | NSMAF, ASAH1 |

### Modulation of the mRNA Expression

Tables 4 and 5 below present the different genes involved respectively in the lipid synthesis or in the cholesterol biosynthetic process which were induced by the tested compound 1. The fold change expresses if they are upregulated (>2) or down regulated (<0.5).

Tables 6 and 7 below present the different genes involved respectively in the lipid synthesis or in the cholesterol biosynthetic process which were induced by the tested compound 2 (7.5 mg/ml). The fold change expresses if they are upregulated (>2) or down regulated (<0.5).

Tables 8 and 9 below present the different genes involved respectively in the lipid synthesis or in the cholesterol biosynthetic process which were induced by the tested compound 3. The fold change expresses if they are upregulated (>2) or down regulated (<0.5)

Tables 10, 11 and 12 below present the different genes involved respectively in the lipid synthesis, in the cholesterol biosynthetic process or in the ceramide metabolic process which were induced by the tested compound 4. The fold change expresses if they are upregulated (>2) or down regulated (<0.5)

TABLE 4
Table of the set of genes involved in the
process of lipid synthesis in NHDF and
stimulated by compound 1 (10 mg/ml) Detection limit < 20; REadj
Relative expression adjusted to the detection limit

| Affy U219 | Control | Compound 1 (10 mg/ml) | | |
|---|---|---|---|---|
| Probe Set ID | REadjl | REadj2 | Fold change | Gene Symbol |
| 11715959_a_at | 160.39 | 519.22 | 3.24 | SREBF1 |
| 11749376_a_at | 31.89 | 65.43 | 2.05 | ABHD4 |
| 11723706_at | 80.75 | 212.72 | 2.63 | ABHD5 |
| 11746009_a_at | 603.21 | 1280.94 | 2.12 | ACAT2 |
| 11717295_s_at | 532.12 | 158.37 | 0.30 | APOD |
| 11756065_x_at | 180.57 | 43.21 | 0.24 | APOD |
| 11732519_at | 168.70 | 60.89 | 0.36 | CH25H |
| 11736285_a_at | 46.25 | 104.91 | 2.27 | CHKA |
| 11736286_a_at | 40.10 | 120.28 | 3.00 | CHKA |
| 11755251_x_at | 380.84 | 1571.10 | 4.13 | FADS1///MIR1908 |
| 11752670_a_at | 617.87 | 2502.62 | 4.05 | FADS1///MIR1908 |
| 11745459_a_at | 389.27 | 1633.20 | 4.20 | FADS1///MIR1908 |
| 11744899_a_at | 45.57 | 120.38 | 2.64 | FADS2 |
| 11744902_a_at | 66.33 | 138.35 | 2.09 | FADS2 |
| 11754939_x_at | 335.14 | 705.50 | 2.11 | FADS2 |
| 11716987_a_at | 277.87 | 875.52 | 3.15 | HMGCS1 |
| 11754918_s_at | 400.21 | 929.88 | 2.32 | HMGCS1 |
| 11720028_x_at | 579.15 | 1468.11 | 2.53 | LDLR |
| 11720029_a_at | 1477.12 | 3123.61 | 2.11 | LDLR |
| 11746974_a_at | 117.24 | 350.27 | 2.99 | LDLR |
| 11748045_x_at | 151.86 | 431.62 | 2.84 | LDLR |
| 11741376_a_at | 31.54 | 93.15 | 2.95 | LRP8 |
| 11722208_a_at | 224.34 | 622.16 | 2.77 | PTGS1 |
| 11722209_a_at | 203.18 | 531.98 | 2.62 | PTGS1 |
| 11722210_a_at | 53.94 | 169.11 | 3.14 | PTGS1 |
| 11749730_a_at | 36.02 | 140.23 | 3.89 | PTGS1 |
| 11729887_at | 216.13 | 498.96 | 2.31 | PTPLA |
| 11715562_x_at | 56.23 | 437.05 | 7.77 | SCD |
| 11715561_s_at | 57.85 | 352.19 | 6.09 | SCD |
| 11715563_s_at | 45.76 | 270.22 | 5.91 | SCD |
| 11756174_s_at | 1367.49 | 3940.97 | 2.88 | SCD |
| 11724014_a_at | 193.96 | 390.22 | 2.01 | SOAT1 |
| 11739503_at | 463.72 | 979.20 | 2.11 | ABCA1 |
| 11719171_a_at | 1752.05 | 3725.47 | 2.13 | AKR1C1 |
| 11761114_x_at | 80.54 | 209.16 | 2.60 | AKR1C2 |
| 11722564_at | 586.61 | 1291.92 | 2.20 | CYP51A1///LRRD1 |
| 11715460_a_at | 769.89 | 1577.01 | 2.05 | DHCR24 |
| 11749042_a_at | 40.29 | 86.50 | 2.15 | DHCR24 |
| 11751337_a_at | 47.72 | 108.18 | 2.27 | DHCR24 |
| 11751338_x_at | 76.80 | 173.84 | 2.26 | DHCR24 |
| 11743808_a_at | 189.13 | 695.09 | 3.68 | DHCR7 |
| 11749332_x_at | 167.80 | 445.85 | 2.66 | EBP |
| 11717859_a_at | 217.05 | 582.73 | 2.68 | EBP |
| 11718366_s_at | 1185.98 | 2391.43 | 2.02 | FDPS |
| 11758249_s_at | 997.21 | 2098.38 | 2.10 | FDPS |
| 11751085_a_at | 149.55 | 451.06 | 3.02 | GBA |
| 11736100_a_at | 162.68 | 445.83 | 2.74 | GBA |
| 11750975_x_at | 163.74 | 439.06 | 2.68 | GBA |
| 11751086_x_at | 143.03 | 338.51 | 2.37 | GBA |
| 11753440_x_at | 96.76 | 229.00 | 2.37 | GBA |
| 11719908_a_at | 144.72 | 423.94 | 2.93 | GBA///GBAP1 |
| 11748292_a_at | 76.66 | 197.15 | 2.57 | GBA///GBAP1 |
| 11751098_a_at | 64.33 | 195.14 | 3.03 | GBA///GBAP1 |
| 11751099_x_at | 107.06 | 294.27 | 2.75 | GBA///GBAP1 |
| 11754819_x_at | 143.04 | 369.80 | 2.59 | GBA///GBAP1 |
| 11753439_a_at | 128.19 | 339.01 | 2.64 | GBA///GBAP1 |
| 11727375_a_at | 196.12 | 395.78 | 2.02 | HMGCR |
| 11757575_x_at | 23.36 | 46.74 | 2.00 | HSD17B14 |
| 11732374_x_at | 37.29 | 91.10 | 2.44 | HSD17B7 |
| 11744474_s_at | 618.41 | 1244.81 | 2.01 | IDI1 |
| 11716337_s_at | 1390.98 | 3419.43 | 2.46 | INSIG1 |
| 11716339_a_at | 362.73 | 1227.18 | 3.38 | INSIG1 |
| 11716338_a_at | 351.80 | 1203.77 | 3.42 | INSIG1 |
| 11730180_s_at | 480.60 | 1037.81 | 2.16 | KDSR |
| 11730181_s_at | 240.11 | 549.40 | 2.29 | KDSR |
| 11751627_a_at | 246.74 | 533.05 | 2.16 | KDSR |
| 11746281_a_at | 78.93 | 182.26 | 2.31 | LPCAT3 |
| 11757008_a_at | 30.62 | 87.56 | 2.86 | LPCAT3 |
| 11755123_x_at | 274.11 | 649.20 | 2.37 | LPCAT3 |
| 11757009_x_at | 48.38 | 101.84 | 2.10 | LPCAT3 |
| 11750566_a_at | 98.01 | 240.89 | 2.46 | LPIN1 |
| 11717620_a_at | 234.42 | 488.74 | 2.08 | LSS |
| 11724199_x_at | 438.97 | 971.54 | 2.21 | LSS |
| 11731324_a_at | 74.56 | 188.43 | 2.53 | LSS |
| 11741012_a_at | 883.63 | 1789.72 | 2.03 | MSMO1 |
| 11729695_a_at | 107.18 | 262.34 | 2.45 | MVD |
| 11747747_a_at | 41.14 | 83.27 | 2.02 | MVK |
| 11719098_a_at | 65.15 | 135.62 | 2.08 | MVK |
| 11715838_a_at | 202.65 | 555.40 | 2.74 | NEU1 |
| 11743916_a_at | 55.34 | 361.48 | 6.53 | NPC1 |
| 11746723_a_at | 42.40 | 192.21 | 4.53 | NPC1 |
| 11747322_s_at | 78.33 | 188.93 | 2.41 | PCYT2 |
| 11724792_a_at | 59.03 | 118.91 | 2.01 | PGS1 |
| 11748428_a_at | 35.07 | 84.52 | 2.41 | PGS1 |
| 11737042_s_at | 40.41 | 120.07 | 2.97 | PHLDB2 |
| 11737039_a_at | 40.22 | 119.06 | 2.96 | PHLDB2 |
| 11737041_a_at | 52.42 | 128.19 | 2.45 | PHLDB2 |
| 11737043_x_at | 72.20 | 178.68 | 2.47 | PHLDB2 |
| 11723546_s_at | 81.71 | 176.07 | 2.15 | PLD1 |

TABLE 5
Table of the set of genes involved in the cholesterol biosynthetic process in
NHDF and stimulated by compound 1 (10 mg/ml)

| Affy U219 | Control | Compound 1 (10 mg/ml) | | |
|---|---|---|---|---|
| Probe Set ID | REadj1 | REadj2 | Fold change | Gene Symbol |
| 11715460_a_at | 769.89 | 1577.01 | 2.05 | DHCR24 |
| 11716114_x_at | 116.70 | 256.70 | 2.20 | POR |
| 11716337_s_at | 1390.98 | 3419.43 | 2.46 | INSIG1 |
| 11717620_a_at | 234.42 | 488.74 | 2.08 | LSS |
| 11718366_s_at | 1185.98 | 2391.43 | 2.02 | FDPS |
| 11743808_a_at | 189.13 | 695.09 | 3.68 | DHCR7 |
| 11722564_at | 586.61 | 1291.92 | 2.20 | CYP51A1///LRRD1 |
| 11716987_a_at | 277.87 | 875.52 | 3.15 | HMGCS1 |
| 11749332_x_at | 167.80 | 445.85 | 2.66 | EBP |
| 11715959_a_at | 160.39 | 519.22 | 3.24 | SREBF1 |
| 11724199_x_at | 438.97 | 971.54 | 2.21 | LSS |
| 11717859_a_at | 217.05 | 582.73 | 2.68 | EBP |
| 11732374_x_at | 37.29 | 91.10 | 2.44 | HSD17B7 |
| 11716339_a_at | 362.73 | 1227.18 | 3.38 | INSIG1 |
| 11716338_a_at | 351.80 | 1203.77 | 3.42 | INSIG1 |
| 11727375_a_at | 196.12 | 395.78 | 2.02 | HMGCR |
| 11758249_s_at | 997.21 | 2098.38 | 2.10 | FDPS |
| 11747747_a_at | 41.14 | 83.27 | 2.02 | MVK |
| 11731324_a_at | 74.56 | 188.43 | 2.53 | LSS |
| 11729695_a_at | 107.18 | 262.34 | 2.45 | MVD |
| 11719098_a_at | 65.15 | 135.62 | 2.08 | MVK |
| 11741012_a_at | 883.63 | 1789.72 | 2.03 | MSMO1 |
| 11744474_s_at | 618.41 | 1244.81 | 2.01 | IDI1 |
| 11748368_x_at | 132.75 | 269.37 | 2.03 | POR |
| 11749042_a_at | 40.29 | 86.50 | 2.15 | DHCR24 |
| 11751337_a_at | 47.72 | 108.18 | 2.27 | DHCR24 |
| 11751338_x_at | 76.80 | 173.84 | 2.26 | DHCR24 |
| 11754918_s_at | 400.21 | 929.88 | 2.32 | HMGCS1 |

TABLE 6
Table of the set of genes involved in the process of lipid synthesis in NHDF
and stimulated by compound 2 (7.5 mg/ml)
Detection limit <20; REadj Relative expression adjusted to the detection limit

| Affy U219 | Control | Compound 2 (7.5 mg/ml) | | |
|---|---|---|---|---|
| Probe Set ID | REadj1 | REadj2 | Fold change | Gene Symbol |
| 11715561_s_at | 138.05 | 277.57 | 2.01 | SCD |
| 11715562_x_at | 149.87 | 302.56 | 2.02 | SCD |
| 11715563_s_at | 116.60 | 241.64 | 2.07 | SCD |
| 11715838_a_at | 227.74 | 495.48 | 2.18 | NEU1 |
| 11729887_at | 256.73 | 516.24 | 2.01 | HACD1 |
| 11748053_x_at | 117.28 | 245.33 | 2.09 | SERINC2 |
| 11748279_s_at | 40.66 | 88.53 | 2.18 | TM7SF2 |
| 11750416_a_at | 668.08 | 1384.05 | 2.07 | TXNRD1 |
| 11751337_a_at | 83.51 | 178.12 | 2.13 | DHCR24 |
| 11752670_a_at | 848.70 | 1734.26 | 2.04 | FADS1 |
| 11755251_x_at | 508.92 | 1114.87 | 2.19 | FADS1 |
| 11756065_x_at | 351.93 | 174.26 | 0.50 | APOD |

TABLE 7
Table of the set of genes involved in the cholesterol biosynthetic process in
NHDF and stimulated by compound 2 (7.5 mg/ml)

| Affy U219 | Control | Compound 2 (7.5 mg/ml) | | |
|---|---|---|---|---|
| Probe Set ID | REadj1 | REadj2 | Fold change | Gene Symbol |
| 11748279_s_at | 40.66 | 88.53 | 2.18 | TM7SF2 |
| 11751337_a_at | 83.51 | 178.12 | 2.13 | DHCR24 |

TABLE 8
Table of the set of genes involved in the process of lipid synthesis in
NHDF and stimulated by compound 3 (10 mg/ml)
Detection limit <20; REadj Relative expression adjusted to the detection
limit

| Affy U219 | Control | Compound 3 (10 mg/ml) | | |
|---|---|---|---|---|
| Probe Set ID | REadj1 | REadj2 | Fold change | Gene Symbol |
| 11715561_s_at | 138.05 | 634.95 | 4.60 | SCD |
| 11715562_x_at | 149.87 | 722.36 | 4.82 | SCD |
| 11715563_s_at | 116.60 | 558.17 | 4.79 | SCD |
| 11715838_a_at | 227.74 | 722.33 | 3.17 | NEU1 |
| 11715959_a_at | 144.81 | 312.15 | 2.16 | SREBF1 |
| 11716292_a_at | 173.61 | 348.08 | 2.00 | G6PD |
| 11716293_x_at | 117.24 | 239.53 | 2.04 | G6PD |
| 11716338_a_at | 465.94 | 1101.68 | 2.36 | INSIG1 |
| 11716339_a_at | 450.00 | 1249.89 | 2.78 | INSIG1 |
| 11716533_a_at | 104.31 | 249.91 | 2.40 | PPT1 |
| 11716987_a_at | 536.29 | 1170.64 | 2.18 | HMGCS1 |
| 11717620_a_at | 415.71 | 1000.50 | 2.41 | LSS |
| 11717859_a_at | 406.76 | 1025.83 | 2.52 | EBP |
| 11718956_a_at | 60.06 | 21.07 | 0.35 | PRKAB2 |
| 11719752_x_at | 50.34 | 105.84 | 2.10 | VAC14 |
| 11719908_a_at | 82.66 | 190.02 | 2.30 | GBA///GBAP1 |
| 11719921_s_at | 87.64 | 209.56 | 2.39 | GM2A |
| 11719923_at | 34.87 | 72.97 | 2.09 | GM2A |
| 11720588_a_at | 71.30 | 154.64 | 2.17 | HSD17B14 |
| 11721046_a_at | 182.33 | 462.86 | 2.54 | SERINC2 |
| 11721665_a_at | 96.54 | 222.24 | 2.30 | PLA2G16 |
| 11724014_a_at | 189.39 | 393.50 | 2.08 | SOAT1 |
| 11725032_a_at | 101.62 | 227.08 | 2.23 | TPP1 |
| 11725116_a_at | 156.55 | 68.44 | 0.44 | NCOR1 |
| 11726265_at | 469.85 | 1130.68 | 2.41 | ME1 |
| 11729681_a_at | 343.21 | 813.79 | 2.37 | PHYH |
| 11729695_a_at | 191.37 | 534.91 | 2.80 | MVD |
| 11729887_at | 256.73 | 674.65 | 2.63 | HACD1 |
| 11731324_a_at | 127.93 | 399.23 | 3.12 | LSS |
| 11736100_a_at | 103.64 | 278.64 | 2.69 | GBA///GBAP1 |
| 11736285_a_at | 54.36 | 137.49 | 2.53 | CHKA |
| 11736286_a_at | 50.87 | 138.86 | 2.73 | CHKA |
| 11737039_a_at | 30.88 | 66.17 | 2.14 | PHLDB2 |
| 11740582_a_at | 593.93 | 1292.95 | 2.18 | ASAH1 |
| 11741376_a_at | 42.14 | 125.76 | 2.98 | LRP8 |
| 11742107_a_at | 129.02 | 289.70 | 2.25 | PPT1 |
| 11743547_a_at | 479.98 | 1038.22 | 2.16 | ASAH1 |
| 11743808_a_at | 372.14 | 1097.58 | 2.95 | DHCR7 |
| 11743916_a_at | 63.03 | 154.34 | 2.45 | NPC1 |
| 11744044_s_at | 111.35 | 239.50 | 2.15 | PCYT2 |
| 11744899_a_at | 119.94 | 263.98 | 2.20 | FADS2 |
| 11745459_a_at | 622.92 | 1891.83 | 3.04 | FADS1 |
| 11746281_a_at | 89.22 | 221.50 | 2.48 | LPCAT3 |
| 11746974_a_at | 150.81 | 357.86 | 2.37 | LDLR |
| 11747178_x_at | 47.62 | 106.68 | 2.24 | GM2A |
| 11747322_s_at | 79.66 | 216.32 | 2.72 | PCYT2 |
| 11748045_x_at | 181.16 | 380.34 | 2.10 | LDLR |
| 11748053_x_at | 117.28 | 342.33 | 2.92 | SERINC2 |
| 11748279_s_at | 40.66 | 91.54 | 2.25 | TM7SF2 |
| 11748292_a_at | 52.08 | 134.21 | 2.58 | GBA///GBAP1 |
| 11748341_a_at | 122.28 | 317.47 | 2.60 | TPP1 |
| 11748342_x_at | 93.45 | 218.65 | 2.34 | TPP1 |
| 11749332_x_at | 366.17 | 907.86 | 2.48 | EBP |
| 11749343_a_at | 29.10 | 66.31 | 2.28 | ABHD4 |
| 11749376_a_at | 32.55 | 69.48 | 2.13 | ABHD4 |
| 11750416_a_at | 668.08 | 1483.80 | 2.22 | TXNRD1 |
| 11750913_a_at | 322.82 | 861.69 | 2.67 | ME1 |
| 11750975_x_at | 107.23 | 250.68 | 2.34 | GBA |
| 11751085_a_at | 93.74 | 266.01 | 2.84 | GBA///GBAP1 |
| 11751086_x_at | 99.46 | 255.53 | 2.57 | GBA |
| 11751098_a_at | 41.72 | 107.13 | 2.57 | GBA///GBAP1 |
| 11751099_x_at | 76.08 | 186.37 | 2.45 | GBAP1 |
| 11751337_a_at | 83.51 | 191.39 | 2.29 | DHCR24 |
| 11751338_x_at | 149.77 | 364.97 | 2.44 | DHCR24 |
| 11751383_a_at | 41.05 | 82.36 | 2.01 | PLA2G15 |
| 11752486_a_at | 70.66 | 224.35 | 3.18 | G6PD |
| 11752670_a_at | 848.70 | 3012.88 | 3.55 | FADS1 |
| 11753248_a_at | 73.10 | 149.69 | 2.05 | TPP1 |
| 11753439_a_at | 78.40 | 184.11 | 2.35 | GBA///GBAP1 |
| 11753440_x_at | 60.55 | 139.49 | 2.30 | GBA |
| 11754819_x_at | 102.52 | 213.06 | 2.08 | GBA///GBAP1 |
| 11755046_a_at | 89.91 | 195.74 | 2.18 | SPHK1 |
| 11755123_x_at | 295.52 | 629.09 | 2.13 | LPCAT3 |
| 11755251_x_at | 508.92 | 1908.34 | 3.75 | FADS1 |
| 11755310_s_at | 229.31 | 462.96 | 2.02 | ACOT1///ACOT2 |
| 11756065_x_at | 351.93 | 154.78 | 0.44 | APOD |
| 11756587_a_at | 431.73 | 1080.74 | 2.50 | PTGDS |
| 11757008_a_at | 29.24 | 70.00 | 2.39 | LPCAT3 |
| 11757469_s_at | 794.20 | 1697.19 | 2.14 | TPP1 |
| 11757575_x_at | 30.55 | 89.08 | 2.92 | HSD17B14 |
| 11762755_x_at | 29.15 | 61.24 | 2.10 | TM7SF2 |

TABLE 9
Table of the set of genes involved in the cholesterol biosynthetic process in
NHDF and stimulated by compound 3 (10 mg/ml)

| Affy U219 | Control | Compound 3 (10 mg/ml) | | |
|---|---|---|---|---|
| Probe Set ID | REadj1 | REadj2 | Fold change | Gene Symbol |
| 11715959_a_at | 144.81 | 312.15 | 2.16 | SREBF1 |
| 11716292_a_at | 173.61 | 348.08 | 2.00 | G6PD |
| 11716293_x_at | 117.24 | 239.53 | 2.04 | G6PD |
| 11716338_a_at | 465.94 | 1101.68 | 2.36 | INSIG1 |
| 11716339_a_at | 450.00 | 1249.89 | 2.78 | INSIG1 |
| 11716987_a_at | 536.29 | 1170.64 | 2.18 | HMGCS1 |
| 11717620_a_at | 415.71 | 1000.50 | 2.41 | LSS |
| 11717859_a_at | 406.76 | 1025.83 | 2.52 | EBP |
| 11729695_a_at | 191.37 | 534.91 | 2.80 | MVD |
| 11731324_a_at | 127.93 | 399.23 | 3.12 | LSS |
| 11743808_a_at | 372.14 | 1097.58 | 2.95 | DHCR7 |
| 11748279_s_at | 40.66 | 91.54 | 2.25 | TM7SF2 |
| 11749332_x_at | 366.17 | 907.86 | 2.48 | EBP |
| 11751337_a_at | 83.51 | 191.39 | 2.29 | DHCR24 |
| 11751338_x_at | 149.77 | 364.97 | 2.44 | DHCR24 |
| 11752486_a_at | 70.66 | 224.35 | 3.18 | G6PD |
| 11762755_x_at | 29.15 | 61.24 | 2.10 | TM7SF2 |

TABLE 10
Table of the set of genes involved in the process of lipid synthesis in
NHDF and stimulated by compound 4 (10 mg/ml)
Detection limit <20; REadj Relative expression adjusted to
the detection limit

| Affy U219 | Control | Compound 4 (10 mg/ml) | | |
|---|---|---|---|---|
| Probe Set ID | REadj1 | REadj2 | Fold change | Gene Symbol |
| 11715440_a_at | 1472.52 | 618.21 | 0.42 | CTGF |
| 11715441_a_at | 2081.87 | 842.74 | 0.40 | CTGF |
| 11715442_s_at | 3150.32 | 1492.26 | 0.47 | CTGF |
| 11715561_s_at | 138.05 | 782.65 | 5.67 | SCD |
| 11715562_x_at | 149.87 | 973.00 | 6.49 | SCD |
| 11715563_s_at | 116.60 | 978.50 | 8.39 | SCD |
| 11715650_a_at | 618.53 | 1753.99 | 2.84 | TXNRD1 |
| 11715837_a_at | 1485.50 | 3015.52 | 2.03 | HEXB |
| 11715838_a_at | 227.74 | 1005.78 | 4.42 | NEU1 |
| 11715959_a_at | 144.81 | 456.21 | 3.15 | SREBF1 |
| 11715994_x_at | 1447.23 | 440.39 | 0.30 | ID2 |
| 11716292_a_at | 173.61 | 438.70 | 2.53 | G6PD |
| 11716293_x_at | 117.24 | 290.47 | 2.48 | G6PD |
| 11716338_a_at | 465.94 | 1272.02 | 2.73 | INSIG1 |
| 11716339_a_at | 450.00 | 1678.67 | 3.73 | INSIG1 |
| 11716533_a_at | 104.31 | 341.22 | 3.27 | PPT1 |
| 11716534_s_at | 169.51 | 440.86 | 2.60 | PPT1 |
| 11716535_a_at | 121.59 | 372.16 | 3.06 | PPT1 |
| 11716718_at | 108.97 | 228.97 | 2.10 | GLA |
| 11716828_a_at | 2174.91 | 4644.23 | 2.14 | NPC2 |
| 11716987_a_at | 536.29 | 1284.34 | 2.39 | HMGCS1 |
| 11717295_s_at | 936.01 | 420.16 | 0.45 | APOD |
| 11717620_a_at | 415.71 | 890.06 | 2.14 | LSS |
| 11717722_s_at | 844.58 | 2142.17 | 2.54 | ASAH1 |
| 11717859_a_at | 406.76 | 1008.77 | 2.48 | EBP |
| 11718255_at | 116.37 | 275.42 | 2.37 | ABHD4 |
| 11718503_a_at | 134.36 | 272.41 | 2.03 | GPCPD1 |
| 11718956_a_at | 60.06 | 20.30 | 0.34 | PRKAB2 |
| 11719323_at | 155.20 | 72.16 | 0.46 | PTEN |
| 11719328_a_at | 151.10 | 381.46 | 2.52 | ALAS1 |
| 11719329_x_at | 131.47 | 369.05 | 2.81 | ALAS1 |
| 11719752_x_at | 50.34 | 122.40 | 2.43 | VAC14 |
| 11719908_a_at | 82.66 | 418.22 | 5.06 | GBA///GBAP1 |
| 11719921_s_at | 87.64 | 296.88 | 3.39 | GM2A |
| 11719922_a_at | 73.24 | 196.89 | 2.69 | GM2A |
| 11719923_at | 34.87 | 91.27 | 2.62 | GM2A |
| 11720588_a_at | 71.30 | 271.08 | 3.80 | HSD17B14 |
| 11720859_s_at | 41.51 | 83.33 | 2.01 | ABHD3 |
| 11721046_a_at | 182.33 | 650.82 | 3.57 | SERINC2 |
| 11721537_a_at | 61.28 | 150.87 | 2.46 | COL4A3BP |
| 11721538_a_at | 402.13 | 880.41 | 2.19 | COL4A3BP |
| 11721665_a_at | 96.54 | 284.35 | 2.95 | PLA2G16 |
| 11722121_a_at | 24.77 | 57.47 | 2.32 | PC |
| 11722564_at | 971.84 | 2314.08 | 2.38 | CYP51A1///LRRD1 |
| 11722982_a_at | 190.59 | 435.41 | 2.28 | LYST |
| 11723499_a_at | 43.13 | 96.03 | 2.23 | PCSK9 |
| 11723988_a_at | 87.54 | 40.94 | 0.47 | NR2F2 |
| 11724013_a_at | 201.96 | 630.86 | 3.12 | SOATI |
| 11724014_a_at | 189.39 | 760.72 | 4.02 | SOATI |
| 11724441_x_at | 604.04 | 180.21 | 0.30 | PTGIS |
| 11725032_a_at | 101.62 | 303.00 | 2.98 | TPP1 |
| 11726265_at | 469.85 | 1412.91 | 3.01 | ME1 |
| 11727275_a_at | 940.90 | 436.67 | 0.46 | KDELC2 |
| 11729681_a_at | 343.21 | 925.21 | 2.70 | PHYH |
| 11729695_a_at | 191.37 | 530.60 | 2.77 | MVD |
| 11729887_at | 256.73 | 648.83 | 2.53 | HACD1 |
| 11730180_s_at | 580.67 | 1233.39 | 2.12 | KDSR |
| 11730181_s_at | 168.08 | 439.49 | 2.61 | KDSR |
| 11731324_a_at | 127.93 | 399.79 | 3.13 | LSS |
| 11732012_x_at | 20.00 | 65.89 | 3.29 | TNFAIP8L3 |
| 11732374_x_at | 68.38 | 166.42 | 2.43 | HSD17B7 |
| 11732432_a_at | 28.83 | 115.36 | 4.00 | GK |
| 11732433_s_at | 27.02 | 128.86 | 4.77 | GK |
| 11733041_a_at | 146.21 | 383.34 | 2.62 | ALAS1 |
| 11733190_a_at | 160.35 | 76.71 | 0.48 | ARSJ |
| 11734201_s_at | 20.00 | 227.60 | 11.38 | GK |
| 11734720_a_at | 24.83 | 66.38 | 2.67 | PLPP2 |
| 11736100_a_at | 103.64 | 526.02 | 5.08 | GBA///GBAP1 |
| 11736285_a_at | 54.36 | 198.99 | 3.66 | CHKA |
| 11736286_a_at | 50.87 | 193.89 | 3.81 | CHKA |
| 11737039_a_at | 30.88 | 101.05 | 3.27 | PHLDB2 |
| 11737040_s_at | 405.82 | 949.43 | 2.34 | PHLDB2 |
| 11737041_a_at | 39.21 | 106.99 | 2.73 | PHLDB2 |
| 11737042_s_at | 30.32 | 73.91 | 2.44 | PHLDB2 |
| 11737043_x_at | 60.62 | 133.27 | 2.20 | PHLDB2 |
| 11739503_at | 191.50 | 680.76 | 3.55 | ABCA1 |
| 11739910_a_at | 69.22 | 160.16 | 2.31 | ELOVL6 |
| 11740580_at | 20.00 | 48.88 | 2.44 | ASAH1 |
| 11740581_s_at | 1006.42 | 2428.54 | 2.41 | ASAH1 |
| 11740582_a_at | 593.93 | 2054.83 | 3.46 | ASAH1 |
| 11741105_a_at | 557.84 | 272.80 | 0.49 | FHL2 |
| 11741376_a_at | 42.14 | 109.99 | 2.61 | LRP8 |
| 11742107_a_at | 129.02 | 434.24 | 3.37 | PPT1 |
| 11742108_a_at | 87.73 | 290.46 | 3.31 | PPT1 |
| 11742745_a_at | 484.99 | 1041.40 | 2.15 | PLIN2 |
| 11742746_a_at | 517.80 | 1152.95 | 2.23 | PLIN2 |
| 11743314_a_at | 143.48 | 441.69 | 3.08 | FASN |
| 11743451_s_at | 125.54 | 258.87 | 2.06 | ELOVL6 |
| 11743547_a_at | 479.98 | 1907.53 | 3.97 | ASAH1 |
| 11743808_a_at | 372.14 | 1204.72 | 3.24 | DHCR7 |
| 11743916_a_at | 63.03 | 441.84 | 7.01 | NPC1 |
| 11744044_s_at | 111.35 | 275.93 | 2.48 | PCYT2 |
| 11744179_x_at | 29.67 | 62.22 | 2.10 | TM7SF2 |
| 11744255_a_at | 1226.17 | 553.57 | 0.45 | ACADVL |
| 11744899_a_at | 119.94 | 361.98 | 3.02 | FADS2 |
| 11744902_a_at | 170.17 | 500.23 | 2.94 | FADS2 |
| 11745276_a_at | 112.92 | 350.47 | 3.10 | SOAT1 |
| 11745459_a_at | 622.92 | 2662.43 | 4.27 | FADS1 |
| 11745736_a_at | 519.02 | 220.73 | 0.43 | ACADVL |
| 11745737_x_at | 1235.02 | 567.83 | 0.46 | ACADVL |
| 11745902_a_at | 1803.41 | 4144.92 | 2.30 | NPC2 |
| 11746281_a_at | 89.22 | 284.98 | 3.19 | LPCAT3 |
| 11746723_a_at | 51.79 | 255.72 | 4.94 | NPC1 |
| 11746863_x_at | 455.38 | 175.21 | 0.38 | ARSJ |
| 11746878_s_at | 1682.91 | 581.09 | 0.35 | ID2 |
| 11746974_a_at | 150.81 | 348.55 | 2.31 | LDLR |
| 11747178_x_at | 47.62 | 155.75 | 3.27 | GM2A |
| 11747186_a_at | 60.68 | 145.04 | 2.39 | PTGES |
| 11747187_x_at | 70.66 | 160.86 | 2.28 | PTGES |
| 11747322_s_at | 79.66 | 277.12 | 3.48 | PCYT2 |
| 11748045_x_at | 181.16 | 401.36 | 2.22 | LDLR |
| 11748053_x_at | 117.28 | 494.04 | 4.21 | SERINC2 |
| 11748279_s_at | 40.66 | 100.08 | 2.46 | TM7SF2 |
| 11748292_a_at | 52.08 | 219.87 | 4.22 | GBA///GBAP1 |
| 11748341_a_at | 122.28 | 483.80 | 3.96 | TPP1 |
| 11748342_x_at | 93.45 | 356.51 | 3.81 | TPP1 |
| 11748855_a_at | 289.27 | 590.60 | 2.04 | CYB5R1 |
| 11749332_x_at | 366.17 | 812.08 | 2.22 | EBP |
| 11749343_a_at | 29.10 | 96.37 | 3.31 | ABHD4 |
| 11749376_a_at | 32.55 | 96.95 | 2.98 | ABHD4 |
| 11749683_a_at | 304.23 | 795.60 | 2.62 | COL4A3BP |
| 11750207_a_at | 501.40 | 1021.58 | 2.04 | HSD17B12 |
| 11750416_a_at | 668.08 | 2045.03 | 3.06 | TXNRD1 |
| 11750566_a_at | 132.38 | 301.54 | 2.28 | LPIN1 |
| 11750720_s_at | 20.90 | 120.18 | 5.75 | GK |
| 11750913_a_at | 322.82 | 1203.10 | 3.73 | ME1 |
| 11750973_a_at | 144.16 | 395.63 | 2.74 | ALAS1 |
| 11750975_x_at | 107.23 | 507.44 | 4.73 | GBA |
| 11751085_a_at | 93.74 | 586.74 | 6.26 | GBA///GBAP1 |
| 11751086_x_at | 99.46 | 419.65 | 4.22 | GBA |
| 11751098_a_at | 41.72 | 200.51 | 4.81 | GBA///GBAP1 |
| 11751099_x_at | 76.08 | 344.54 | 4.53 | GBAP1 |
| 11751337_a_at | 83.51 | 176.26 | 2.11 | DHCR24 |
| 11751338_x_at | 149.77 | 345.60 | 2.31 | DHCR24 |
| 11751383_a_at | 41.05 | 103.53 | 2.52 | PLA2G15 |
| 11751627_a_at | 164.00 | 463.10 | 2.82 | KDSR |
| 11752486_a_at | 70.66 | 220.41 | 3.12 | G6PD |
| 11752670_a_at | 848.70 | 3882.61 | 4.57 | FADS1 |
| 11752817_s_at | 267.36 | 777.41 | 2.91 | TPP1 |
| 11752885_x_at | 118.11 | 318.95 | 2.70 | TPP1 |
| 11753248_a_at | 73.10 | 234.83 | 3.21 | TPP1 |
| 11753249_x_at | 182.63 | 492.94 | 2.70 | TPP1 |
| 11753439_a_at | 78.40 | 357.99 | 4.57 | GBA///GBAP1 |
| 11753440_x_at | 60.55 | 292.80 | 4.84 | GBA |
| 11754526_a_at | 232.96 | 605.82 | 2.60 | PTGES |
| 11754812_a_at | 160.00 | 46.34 | 0.29 | NPAS2 |
| 11754819_x_at | 102.52 | 374.98 | 3.66 | GBA///GBAP1 |
| 11754939_x_at | 500.54 | 1128.95 | 2.26 | FADS2 |
| 11755046_a_at | 89.91 | 349.35 | 3.89 | SPHK1 |
| 11755123_x_at | 295.52 | 715.20 | 2.42 | LPCAT3 |
| 11755251_x_at | 508.92 | 2235.51 | 4.39 | FADS1 |
| 11755396_a_at | 54.49 | 177.48 | 3.26 | PLA2G15 |
| 11756065_x_at | 351.93 | 133.10 | 0.38 | APOD |
| 11756587_a_at | 431.73 | 1259.53 | 2.92 | PTGDS |
| 11756724_x_at | 146.33 | 407.92 | 2.79 | PPT1 |
| 11757008_a_at | 29.24 | 91.45 | 3.13 | LPCAT3 |
| 11757009_x_at | 41.82 | 120.51 | 2.88 | LPCAT3 |
| 11757184_a_at | 260.48 | 537.03 | 2.06 | SC5D |
| 11757469_s_at | 794.20 | 2340.47 | 2.95 | TPP1 |
| 11757575_x_at | 30.55 | 162.71 | 5.33 | HSD17B14 |
| 11757824_s_at | 361.74 | 180.66 | 0.50 | RGL1 |
| 11758133_s_at | 202.08 | 411.42 | 2.04 | COL4A3BP |

TABLE 11
Table of the set of genes involved in the cholesterol biosynthetic process
in NHDF and stimulated by compound 4 (10 mg/ml)

| Affy U219 | Control | Compound 4 (10 mg/ml) | | |
|---|---|---|---|---|
| Probe Set ID | REadj1 | REadj2 | Fold change | Gene Symbol |
| 11715959_a_at | 144.81 | 456.21 | 3.15 | SREBF1 |
| 11716114_x_at | 112.82 | 414.95 | 3.68 | POR |
| 11716292_a_at | 173.61 | 438.70 | 2.53 | G6PD |
| 11716293_x_at | 117.24 | 290.47 | 2.48 | G6PD |
| 11716338_a_at | 465.94 | 1272.02 | 2.73 | INSIG1 |
| 11716339_a_at | 450.00 | 1678.67 | 3.73 | INSIG1 |
| 11716987_a_at | 536.29 | 1284.34 | 2.39 | HMGCS1 |
| 11717620_a_at | 415.71 | 890.06 | 2.14 | LSS |
| 11717859_a_at | 406.76 | 1008.77 | 2.48 | EBP |
| 11722564_at | 971.84 | 2314.08 | 2.38 | CYP51A1///LRRD1 |
| 11729695_a_at | 191.37 | 530.60 | 2.77 | MVD |
| 11731324_a_at | 127.93 | 399.79 | 3.13 | LSS |
| 11732374_x_at | 68.38 | 166.42 | 2.43 | HSD17B7 |
| 11743808_a_at | 372.14 | 1204.72 | 3.24 | DHCR7 |
| 11744179_x_at | 29.67 | 62.22 | 2.10 | TM7SF2 |
| 11746973_x_at | 28.89 | 93.09 | 3.22 | POR |
| 11748279_s_at | 40.66 | 100.08 | 2.46 | TM7SF2 |
| 11748368_x_at | 114.58 | 386.63 | 3.37 | POR |
| 11749332_x_at | 366.17 | 812.08 | 2.22 | EBP |
| 11751337_a_at | 83.51 | 176.26 | 2.11 | DHCR24 |
| 11751338_x_at | 149.77 | 345.60 | 2.31 | DHCR24 |
| 11752486_a_at | 70.66 | 220.41 | 3.12 | G6PD |
| 11757184_a_at | 260.48 | 537.03 | 2.06 | SC5D |
| 11757517_x_at | 145.26 | 314.19 | 2.16 | POR |

TABLE 12
Table of the set of genes involved in the ceramide metabolic process
in NHDF and stimulated by compound 4 (10 mg/ml)

| Affy U219 | Control | Compound 4 (10 mg/ml) | | |
|---|---|---|---|---|
| Probe Set ID | REadj1 | REadj2 | Fold change | Gene Symbol |
| 11717214_s_at | 684.75 | 1378.59 | 2.01 | NSMAF |
| 11717722_s_at | 844.58 | 2142.17 | 2.54 | ASAH1 |
| 11721537_a_at | 61.28 | 150.87 | 2.46 | COL4A3BP |
| 11721538_a_at | 402.13 | 880.41 | 2.19 | COL4A3BP |
| 11726630_a_at | 122.50 | 252.64 | 2.06 | HTRA2 |
| 11731408_x_at | 73.38 | 273.96 | 3.73 | CLN3 |
| 11740559_a_at | 94.47 | 221.42 | 2.34 | NSMAF |
| 11740560_x_at | 191.85 | 420.15 | 2.19 | NSMAF |
| 11740580_at | 20.00 | 48.88 | 2.44 | ASAH1 |
| 11740581_s_at | 1006.42 | 2428.54 | 2.41 | ASAH1 |
| 11740582_a_at | 593.93 | 2054.83 | 3.46 | ASAH1 |
| 11743547_a_at | 479.98 | 1907.53 | 3.97 | ASAH1 |
| 11744874_x_at | 52.01 | 206.83 | 3.98 | CLN3 |
| 11747203_x_at | 52.77 | 156.70 | 2.97 | CLN3 |
| 11747739_a_at | 54.10 | 126.13 | 2.33 | PPP2R1A |
| 11747740_x_at | 198.82 | 529.31 | 2.66 | PPP2R1A |
| 11748869_a_at | 194.83 | 631.60 | 3.24 | CLN3 |
| 11748870_x_at | 124.01 | 413.38 | 3.33 | CLN3 |
| 11749683_a_at | 304.23 | 795.60 | 2.62 | COL4A3BP |
| 11751383_a_at | 41.05 | 103.53 | 2.52 | PLA2G15 |
| 11753053_x_at | 71.23 | 219.67 | 3.08 | CLN3 |
| 11755396_a_at | 54.49 | 177.48 | 3.26 | PLA2G15 |
| 11756016_x_at | 144.81 | 405.19 | 2.80 | CLN3 |
| 11756421_x_at | 105.74 | 227.59 | 2.15 | HTRA2 |
| 11758133_s_at | 202.08 | 411.42 | 2.04 | COL4A3BP |

### Analysis of Signaling Pathway

A more advanced bioinformatics analysis was performed using the Ingenuity Pathway Analysis software (IPA from Qiagen®). This analysis allows the identification of the impacted signaling pathways and predicts their modulation.

TABLE 13
modulation of the lipid synthesis by compound 1 (10 mg/ml) on NHDF

| Gene symbol | Probe set ID | Fold change |
|---|---|---|
| ABCA1 | 11739503_at | 2.112 |
| ABHD5 | 11723706_at | 2.634 |
| ACAT2 | 11746009_a_at | 2.124 |
| ACSS2 | 11722346_a_at | 2.288 |
| ADORA2B | 11741056_a_at | 2.461 |
| AKR1B1 | 11715430_a_at | 4.194 |
| AKR1C1/AKR1C2 | 11761114_x_at | 2.597 |
| ALDH1A3 | 11750102_x_at | −3.504 |
| ANGPT1 | 11726692_at | −2.510 |
| CD9 | 11746940_a_at | 2.337 |
| CH25H | 11732519_at | −2.770 |
| CHKA | 11736286_a_at | 2.999 |
| CLN3 | 11744874_x_at | 2.570 |
| CYP19A1 | 11715729_s_at | 2.736 |
| CYP51A1 | 11722564_at | 2.202 |
| DHCR7 | 11743808_a_at | 3.675 |
| DHCR24 | 11751337_a_at | 2.267 |
| EBP | 11717859_a_at | 2.685 |
| EGR1 | 11752940_a_at | −2.928 |
| FADS1 | 11745459_a_at | 4.196 |
| FADS2 | 11744899_a_at | 2.642 |
| FDPS | 11758249_s_at | 2.104 |
| FGF2 | 11725040_at | 2.008 |
| FOS | 11734659_a_at | −2.521 |
| GBA | 11751085_a_at | 3.016 |
| GPER1 | 11753609_x_at | 2.031 |
| GRP | 11756874_a_at | −2.699 |
| HMGCR | 11727375_a_at | 2.018 |
| HMOX1 | 11753446_x_at | 5.168 |
| HSD17B7 | 11732374_x_at | 2.443 |
| HSD3B7 | 11734172_a_at | 2.132 |
| HTR2B | 11728104_at | 2.118 |
| IDI1 | 11744474_s_at | 2.013 |
| IGFBP2 | 11722583_a_at | −2.717 |
| INSIG1 | 11716338_a_at | 3.422 |
| KDSR | 11730181_s_at | 2.288 |
| LDLR | 11746974_a_at | 2.988 |
| LPIN1 | 11750566_a_at | 2.458 |
| LSS | 11731324_a_at | 2.527 |
| MAP3K8 | 11736217_at | 3.713 |
| ME1 | 11726265_at | 2.548 |
| MVD | 11729695_a_at | 2.448 |
| MVK | 11719098_a_at | 2.082 |
| NGF | 11729937_at | −2.648 |
| NPC1 | 11743916_a_at | 6.532 |
| NRG1 | 11742478_a_at | −2.111 |
| NSMAF | 11740559_a_at | 2.624 |
| NTN1 | 11730467_at | 2.832 |
| PCYT2 | 11747322_s_at | 2.412 |
| PDE5A | 11720579_a_at | −3.902 |
| PDK4 | 11716974_a_at | −2.280 |
| PGS1 | 11748428_a_at | 2.410 |
| PLAU | 11717154_a_at | −2.785 |
| PLD1 | 11723546_s_at | 2.155 |
| POR | 11716114_x_at | 2.200 |
| PTGS1 | 11749730_a_at | 3.893 |
| RAB27A | 11758934_x_at | 2.279 |
| RUNX1 | 11742191_a_at | 2.441 |
| S1PR1 | 11743816_s_at | 2.651 |
| SCD | 11715562_x_at | 7.772 |
| SEMA3A | 11730391_at | −3.169 |
| SERINC2 | 11748053_x_at | 2.635 |
| SLC1A3 | 11726252_a_at | 4.715 |
| SOAT1 | 11724014_a_at | 2.012 |
| SOCS3 | 11719218_at | 3.734 |
| SREBF1 | 11715959_a_at | 3.237 |
| ST6GALNAC5 | 11727448_x_at | 2.080 |
| STC1 | 11750279_a_at | 3.016 |

TABLE 14
modulation of the cholesterol biosynthetic process by
compound 1 (10 mg/ml) on NHDF

| Gene symbol | Probe set ID | Fold change |
|---|---|---|
| CH25H | 11732519_at | −2.770 |
| SOAT1 | 11724014_a_at | 2.012 |
| LSS | 11731324_a_at | 2.527 |
| FDPS | 11758249_s_at | 2.104 |
| HSD17B7 | 11732374_x_at | 2.443 |
| POR | 11716114_x_at | 2.200 |
| SREBF1 | 11715959_a_at | 3.237 |
| HMGCR | 11727375_a_at | 2.018 |
| LDLR | 11746974_a_at | 2.988 |
| NPC1 | 11743916_a_at | 6.532 |
| DHCR24 | 11751337_a_at | 2.267 |
| INSIG1 | 11716338_a_at | 3.422 |
| EBP | 11717859_a_at | 2.685 |
| IDI1 | 11744474_s_at | 2.013 |
| MVK | 11719098_a_at | 2.082 |
| CYP51A1 | 11722564_at | 2.202 |
| DHCR7 | 11743808_a_at | 3.675 |

TABLE 15
modulation of signalling pathway by compound 1 (10 mg/ml) on NHDF
The modulation is a stimulation when the Activation z-score is a positive value and an
inhibition when the Activation z-score is a negative value.

| Diseases or | | | |
|---|---|---|---|
| Functions | | Activation z- | |
| Annotation | p-Value | score | Molecules |
| synthesis of | 6.8E−16 | 2.668 | ABCA1, ABHD5, ACAT2, ACSS2, |
| lipid | | | ADORA2B, AKR1B1, AKR1C1/AKR1C2, |
| | | | ALDH1A3, ANGPT1, CD9, CH25H, |
| | | | CHKA, CLN3, CYP19A1, CYP51A1, |
| | | | DHCR24, DHCR7, EBP, EGR1, FADS1, |
| | | | FADS2, FDPS, FGF2, FOS, GBA, GPER1, |
| | | | GRP, HMGCR, HMOX1, HSD17B7, |
| | | | HSD3B7, HTR2B, IDI1, IGFBP2, INSIG1, |
| | | | KDSR, LDLR, LPIN1, LSS, MAP3K8, |
| | | | ME1, MVD, MVK, NGF, NPC1, NRG1, |
| | | | NSMAF, NTN1, PCYT2, PDE5A, PDK4, |
| | | | PGS1, PLAU, PLD1, POR, PTGS1, |
| | | | RAB27A, RUNX1, S1PR1, SCD, |
| | | | SEMA3A, SERINC2, SLC1A3, SOAT1, |
| | | | SOCS3, SREBF1, ST6GALNAC5, STC1 |
| synthesis of | 2.51E−13 | 2.169 | CH25H, CYP51A1, DHCR24, DHCR7, |
| cholesterol | | | EBP, FDPS, HMGCR, HSD17B7, IDI1, |
| | | | INSIG1, LDLR, LSS, MVK, NPC1, POR, |
| | | | SOAT1, SREBF1 |

The analysis of signaling pathways has shown a predictive activation of the lipid synthesis and the cholesterol biosynthetic process at a transcriptional level by compound 1.

Thus, under the experimental conditions of the assay, the treatment of NHDF with compound 1, tested at 10 mg/ml, resulted in an up regulation of lipid and cholesterol synthesis.

TABLE 16
modulation of signalling pathway by compound 3 (10 mg/ml) on NHDF
The modulation is a stimulation when the Activation z-score is a positive value and an
inhibition when the Activation z-score is a negative value.

| Diseases or | | | |
|---|---|---|---|
| Functions | | Activation | |
| Annotation | p-value | z-score | Molecules |
| Synthesis of | 1.46E−14 | 1.002 | ACSS2, ALDH1A3, ASAH1, BHLHE40, |
| lipid | | | CD9, CHKA, CLN3, DHCR24, DHCR7, |
| | | | EBF1, EBP, EGR1, ETV1, FADS1, FADS2, |
| | | | FOS, G6PD, GBA, GREM2, GRP, HACD1, |
| | | | HMGCS1, HMOX1, HSD17B14, HSD3B7, |
| | | | HTR2B, IL6, INSIG1, LDLR, LSS, |
| | | | MAP3K8, ME1, MVD, NFIL3, NGF, |
| | | | NPC1, NR4A3, PCYT2, PDE5A, PLAAT3, |
| | | | PPT1, PRKAB2, PTGDS, RUNX1, SCD, |
| | | | SERINC2, SLC1A3, SOAT1, SPHK1, |
| | | | SPP1, SREBF1, STC1, TCF7L2, TM7SF2, |
| | | | TMEM38B, TRERF1, VAC14 |
| Metabolism | 9.13E−12 | 1.083 | DHCR24, DHCR7, EBP, G6PD, GBA, |
| of cholesterol | | | HMGCS1, HSD3B7, INSIG1, LDLR, LSS, |
| | | | MVD, NPC1, SCD, SOAT1, SPP1, |
| | | | SREBF1, TM7SF2, TNFSF4 |
| Synthesis of | 1.67E−11 | 0.399 | DHCR24, DHCR7, EBP, G6PD, HMGCS1, |
| cholesterol | | | INSIG1, LDLR, LSS, MVD, NPC1, SCD, |
| | | | SOAT1, SPP1, SREBF1, TM7SF2 |

TABLE 17
modulation of signalling pathway by compound 4 (10 mg/ml) on NHDF
The modulation is a stimulation when the Activation z-score is a positive value and an
inhibition when the Activation z-score is a negative value.

| Diseases or | | | |
|---|---|---|---|
| Functions | | Activation | |
| Annotation | p-value | z-score | Molecules |
| Fatty acid | 4.02E−10 | 1.958 | ABCA1, ACADVL, ACSS2, AKR1B1, |
| metabolism | | | ASAH1, ATP10D, CAV2, CCL2, CD9, |
| | | | CEBPB, CERT1, CHKA, CSF1, CYP27A1, |
| | | | DAB1, DBI, DLAT, EDNRA, EGR1, |
| | | | ELOVL6, EPHX1, FABP3, FADS1, |
| | | | FADS2, FASN, GBA, GDF15, GM2A, |
| | | | HACD1, HMOX1, HTR2B, IL15, IL6, |
| | | | INSIG1, IRAKI, LDLR, LEPR, LPIN1, |
| | | | LSS, ME1, MGST2, MITF, MMP3, NGF, |
| | | | NPC1, NPC2, NRG1, OSBPL8, PCOLCE2, |
| | | | PDPN, PER2, PHGDH, PITPNC1, |
| | | | PLA2G15, PLIN2, POR, PPT1, PRKAB2, |
| | | | PTCH1, PTGDS, PTGES, PTGIS, RAC1, |
| | | | RUNX1, S1PR1, S1PR3, SC5D, SCD, |
| | | | SEMA3A, SLC1A3, SLC9A3R1, SMAD3, |
| | | | SNTB1, SOAT1, SPHK1, SREBF1, SSPN, |
| | | | TNFAIP8L3, TXN, WNT5A |
| Synthesis of | 1.30E−08 | 1.371 | ABCA1, ACADVL, ACSS2, AKR1B1, |
| fatty acid | | | ASAH1, CAV2, CEBPB, CSF1, CYP27A1, |
| | | | DAB1, DLAT, EDNRA, EGR1, ELOVL6, |
| | | | FADS1, FADS2, FASN, HACD1, HMOX1, |
| | | | HTR2B, IL15, IL6, INSIG1, LDLR, LEPR, |
| | | | MGST2, MITF, NGF, NPC1, NPC2, NRG1, |
| | | | PPT1, PRKAB2, PTGDS, PTGES, PTGIS, |
| | | | RAC1, RUNX1, S1PR1, S1PR3, SCD, |
| | | | SEMA3A, SLC1A3, SMAD3, SOAT1, |
| | | | SPHK1, SREBF1, SSPN, TXN |
| Differentiation | 1.16E−09 | 0.354 | CAVIN1, CCND1, CEBPB, CMKLR1, |
| of | | | CREB5, DIO2, EBF1, EGR2, FABP3, |
| adipocytes | | | HDAC9, HMOX1, ID2, IL6, INSIG1, |
| | | | JAG1, KLF4, LAMA4, LPIN1, METRNL, |
| | | | MMP1, MMP11, MMP3, NFIA, NR4A1, |
| | | | NR4A3, NRG1, OSBPL8, PER2, PLAUR, |
| | | | PTGDS, RORA, RUNX1T1, SCD, |
| | | | SEMA3A, SFRP2, SH3PXD2B, SMAD3, |
| | | | SOD2, SPP1, SREBF1, TCF7L2, TIMP3, |
| | | | TRIB2, VGLL3, WNT5A |
| Synthesis of | 3.28E−16 | 1.730 | ABCA1, ABHD3, ACADVL, ACSS2, |
| lipid | | | AGTR1, AHR, AKR1B1, ALDH1A3, |
| | | | ANGPT1, ASAH1, BHLHE40, CAV2, |
| | | | CCL2, CD9, CEBPB, CERT1, CHKA, |
| | | | CLN3, CSF1, CYP27A1, CYP51A1, DAB1, |
| | | | DBI, DENND1A, DHCR24, DHCR7, |
| | | | DLAT, EBF1, EBP, EDNRA, EGR1, |
| | | | ELOVL6, ETV1, FADS1, FADS2, FASN, |
| | | | FOS, FOSL1, G6PD, GBA, GDF15, GK, |
| | | | GLA, GRP, HACD1, HEXB, HMGCS1, |
| | | | HMOX1, HSD17B14, HSD17B7, HSD3B7, |
| | | | HSPA5, HTR2B, IGFBP2, IL15, IL6, |
| | | | INSIG1, KDSR, KLF4, LDLR, LEPR, |
| | | | LPIN1, LSS, ME1, MGST2, MITF, MVD, |
| | | | NFIL3, NGF, NPC1, NPC2, NR2F2, |
| | | | NR3C1, NR4A1, NR4A3, NRG1, NSMAF, |
| | | | PCYT2, PDE5A, PFKFB2, PITPNM3, |
| | | | PLA2R1, PLAAT3, PLIN2, PLPP2, POR, |
| | | | PPT1, PRKAB2, PRPF19, PTEN, PTGDS, |
| | | | PTGES, PTGIS, PTPN13, RAB27A, RAC1, |
| | | | RGS3, RUNX1, S1PR1, S1PR3, SC5D, |
| | | | SCD, SEMA3A, SERINC2, SLC1A3, |
| | | | SLC9A3R1, SMAD3, SOAT1, SPHK1, |
| | | | SPP1, SREBF1, SSPN, STC1, TCF7L2, |
| | | | THRB, TM7SF2, TMEM38B, TRPV2, |
| | | | TXN, VAC14, WNT5A |

The analysis of signaling pathways has shown a predictive activation of the lipid synthesis and the cholesterol biosynthetic process at a transcriptional level by compound 3 (10 mg/ml), and a predictive activation of the fatty acid and lipid synthesis and a stimulation of the adipocytes differentiation by compound 4 (10 mg/ml)

Thus, under the experimental conditions of the assay, the treatment of NHDF with test compound 3 or 4 resulted in an up regulation of lipids synthesis, including fatty acids, cholesterol or ceramides.

1-22. (canceled)

23. A method for skin plumping, skin volumizing, skin densifying, wrinkle filling, skin or hair moisturizing, skin or hair relipiding, or stimulating hair growth comprising the administration to a person in need thereof of an effective amount of a glycopeptide of the following formula I: 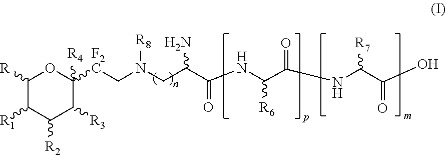
or a tautomer thereof, a stereoisomer thereof, a mixture of stereoisomers thereof in any proportions, a physiologically acceptable salt thereof, a solvate thereof, or a combination thereof, in which:
n represents an integer from 1 to 6,
m represents 0 or 1,
p represents 0 or 1
R represents H, F, CH₃, CH₂F, or CH₂OH,
R₁, R₂ and R₃ represent, independently from one another, H, F, or OH,
R₄ represents a hydrogen, a halogen, or OH,
R₆ and R₇ represent, independently from each other, a hydrogen, a (C₁-C₆)alkyl, an aryl, or an aryl-(C₁-C₆)alkyl, and
R₈ represents H or a CO-(C₁-C₂₀)alkyl.

24. The method according to claim 23, wherein the glycopeptide is a glycopeptide of the following formula (Ia), (Ib) or (Ic): 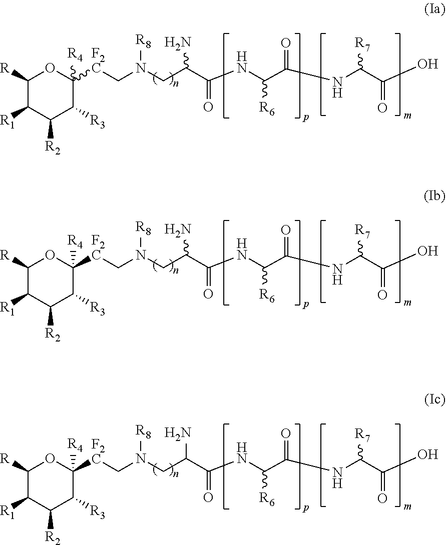
or a tautomer thereof, a physiologically acceptable salt thereof, a solvate thereof, or a combination thereof.

25. The method according to claim 23, wherein n represents an integer from 2 to 6.

26. The method according to claim 23, wherein R represents CH₂OH, and R₁, R₂ and R₃ each represent OH.

27. The method according to claim 26, wherein R₄ represents OH.

28. The method according to claim 23, wherein R₆ and R₇ represent, independently from each other, a (C₁-C₆)alkyl.

29. The method according to claim 23, wherein the glycopeptide is chosen from: 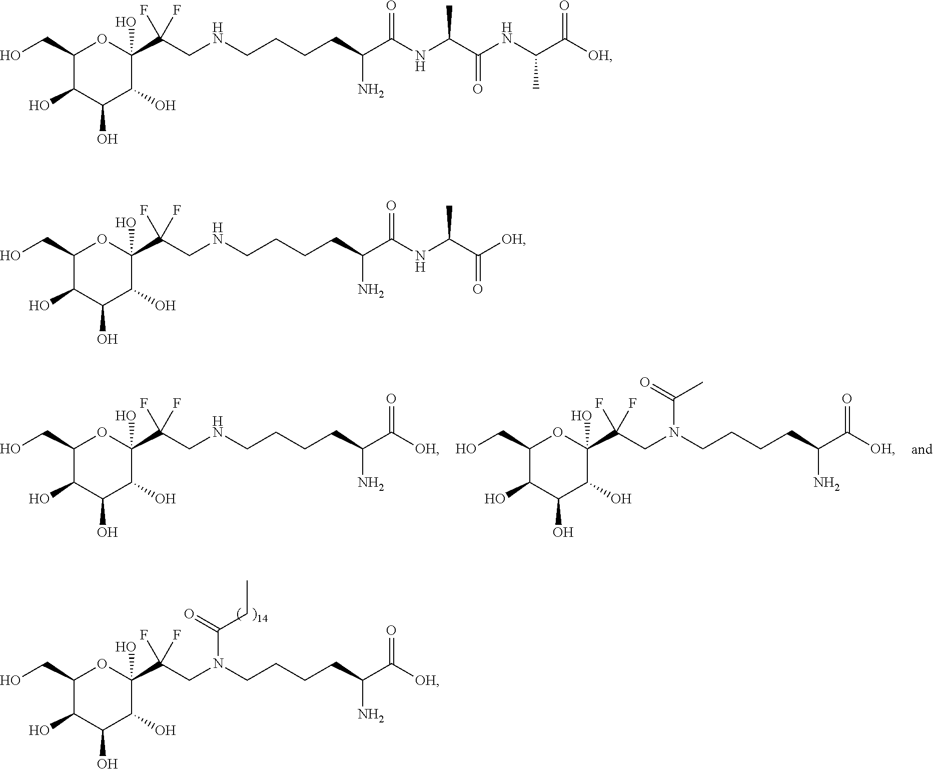
of is a tautomer thereof, a physiologically acceptable salt thereof, a solvate thereof or a combination thereof.

30. A method for skin plumping, skin volumizing, skin densifying, wrinkle filling, skin or hair moisturizing, skin or hair relipiding, or stimulating hair growth comprising the administration to a person in need thereof of an effective amount of a cosmetic or dermatological composition comprising at least one physiologically acceptable excipient and a glycopeptide of the following formula I: 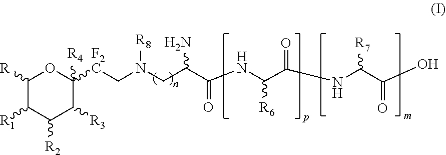
or a tautomer thereof, a stereoisomer thereof, a mixture of stereoisomers thereof in any proportions, a physiologically acceptable salt thereof, a solvate thereof, or a combination thereof, in which:
n represents an integer from 1 to 6,
m represents 0 or 1,
p represents 0 or 1
R represents H, F, CH₃, CH₂F, or CH₂OH,
R₁, R₂ and R₃ represent, independently from one another, H, F, or OH,
R₄ represents a hydrogen, a halogen, or OH,
R₆ and R₇ represent, independently from each other, a hydrogen, a (C₁-C₆)alkyl, an aryl, or an aryl-(C₁-C₆)alkyl, and
R₈ represents H or a CO-(C₁-C₂₀)alkyl.

31. A method for treating dry skin, atopic dermatitis, atopic eczema or psoriasis comprising the administration to a person in need thereof of an effective amount of a glycopeptide of the following formula I, 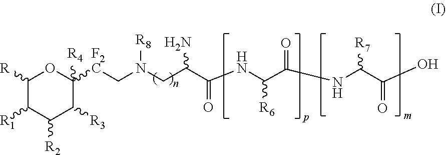
or a tautomer thereof, a stereoisomer thereof, a mixture of stereoisomers thereof in any proportions, a physiologically acceptable salt thereof, a solvate thereof, or a combination thereof, in which:
n represents an integer from 1 to 6,
m represents 0 or 1,
p represents 0 or 1
R represents H, F, CH₃, CH₂F, or CH₂OH,
R₁, R₂ and R₃ represent, independently from one another, H, F, or OH,
R₄ represents a hydrogen, a halogen, or OH,
R₆ and R₇ represent, independently from each other, a hydrogen, a (C₁-C₆)alkyl, an aryl, or an aryl-(C₁-C₆)alkyl, and
R₈ represents H or a CO-(C₁-C₂₀)alkyl.

32. The method according to claim 31, wherein the glycopeptide is a glycopeptide of the following formula (Ia), (Ib) or (Ic): 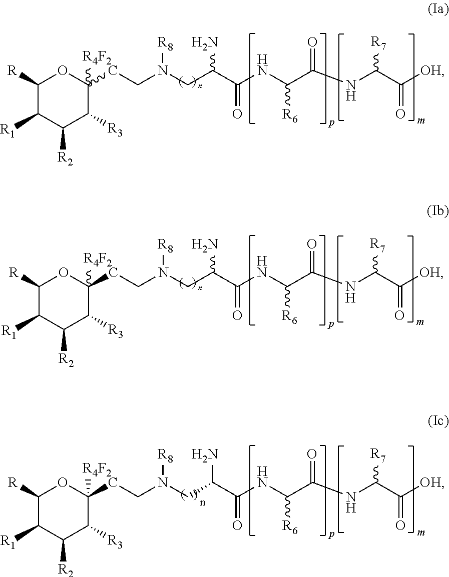
or a tautomer thereof, a physiologically acceptable salt thereof, a solvate thereof, or a combination thereof.

33. The method according to claim 31, wherein n represents an integer from 2 to 6.

34. The method according to claim 31, wherein R represents CH₂OH, R₁, R₂ and R₃ each represent OH.

35. The method according to claim 34, wherein R₄ represents OH.

36. The method according to claim 31, wherein R₆ and R₇ represent, independently from each other, a (C₁-C₆)alkyl.

37. The method according to claim 31, wherein the glycopeptide is chosen among: 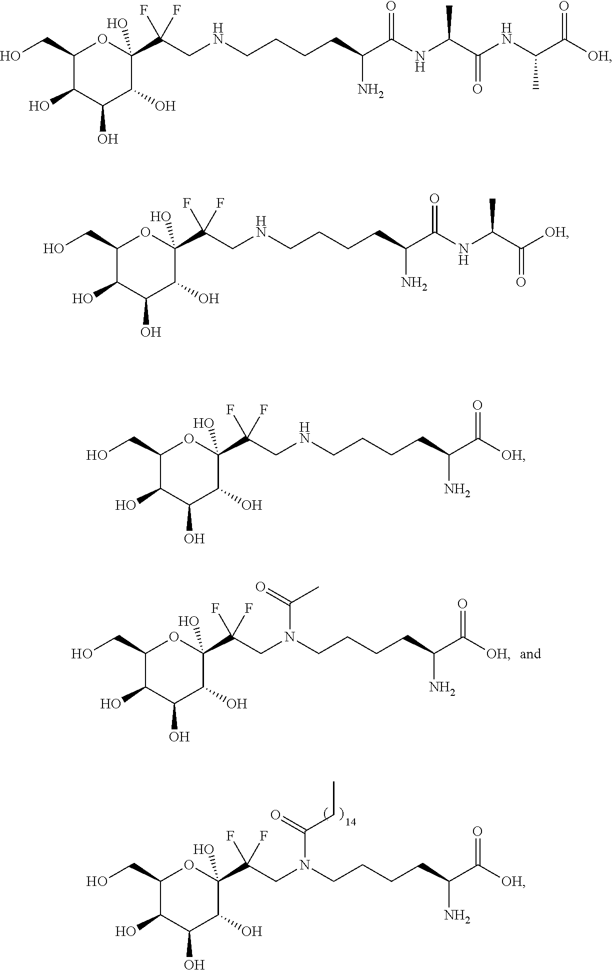
or is a tautomer thereof, a physiologically acceptable salt thereof, a solvate thereof, or a combination thereof.

38. A method for treating dry skin, atopic dermatitis, atopic eczema or psoriasis comprising the administration to a person in need thereof of an effective amount of a cosmetic or dermatological composition comprising a glycopeptide at least one physiologically acceptable excipient and a glycopeptide of the following formula I. 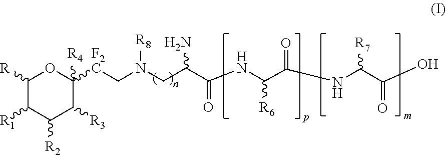
or a tautomer thereof, a stereoisomer thereof, a mixture of stereoisomers thereof in any proportions, a physiologically acceptable salt thereof, a solvate thereof, or a combination thereof, in which:
n represents an integer from 1 to 6,
m represents 0 or 1,
p represents 0 or 1
R represents H, F, CH₃, CH₂F, or CH₂OH,
R₁, R₂ and R₃ represent, independently from one another, H, F, or OH,
R₄ represents a hydrogen, a halogen, or OH,
R₆ and R₇ represent, independently from each other, a hydrogen, a (C₁-C₆)alkyl, an aryl, or an aryl-(C₁-C₆)alkyl, and
R₈ represents H or a CO-(C₁-C₂₀)alkyl.

39. A glycopeptide of the following formula I″: 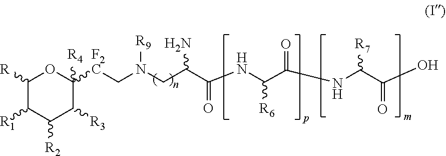
or a tautomer thereof, a stereoisomer thereof, a mixture of stereoisomers thereof in any proportions, a physiologically acceptable salt thereof, a solvate thereof, or a combination thereof, in which:
n represents an integer from 1 to 6,
m represents 0 or 1,
p represents 0 or 1
R represents H, F, CH₃, CH₂F, or CH₂OH,
R₁, R₂ and R₃ represent, independently from one another, H, F, or OH,
R₄ represents a hydrogen, a halogen, or OH,
R₆ and R₇ represent, independently from each other, a hydrogen, a (C₁-C₆)alkyl, an aryl, or an aryl-(C₁-C₆)alkyl, and
R₉ represents a CO-(C₁-C₂₀)alkyl.

40. The glycopeptide according to claim 39, having the following formula (Ia“), (Ib”) or (IC″): 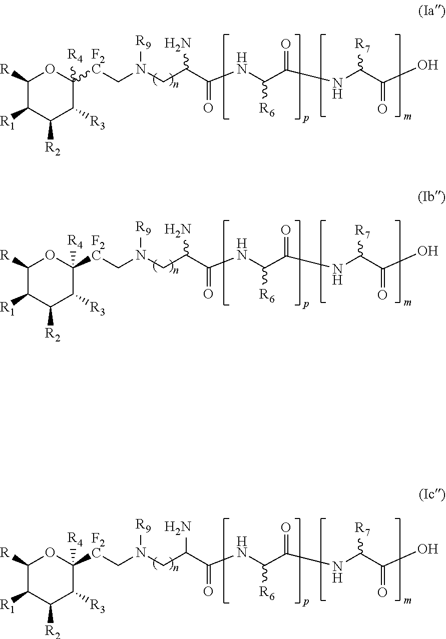
or a tautomer thereof, a physiologically acceptable salt thereof, a solvate thereof, or a combination thereof.

41. The glycopeptide according to claim 39, wherein n represents an integer from 2 to 6.

42. The glycopeptide according to claim 39, wherein R represents CH₂OH, R₁, R₂ and R₃ each represent OH.

43. The glycopeptide according to claim 42, wherein R₄ represents OH.

44. The glycopeptide according to claim 39, wherein R₆ and R₇ represent, independently from each other, a (C₁-C₆)alkyl.

45. The glycopeptide according to claim 39, being chosen from: 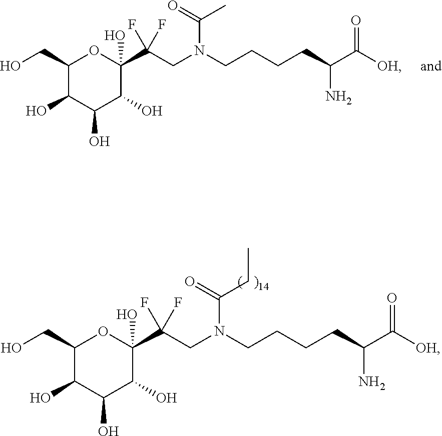
or being a tautomer thereof, a physiologically acceptable salt thereof, a solvate thereof, or a combination thereof.

46. A cosmetic or dermatological composition comprising a glycopeptide according to claim 39 and at least one physiologically acceptable excipient.
